(19) United States
(12) Patent Application Publication
(10) Pub. No.: US 2006/0127893 A1
(43) Pub. Date: Jun. 15, 2006

(54) Modification of human variable domains

(76) Inventors: Stefan Ewert, Zurich (CH); Thomas Huber, Schaffhausen (CH); Annemarie Honegger, Zurich (CH); Andreas Pluckthun, Zurich (CH)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/484,105

(22) PCT Filed: Jul. 19, 2002
(86) PCT No.: PCT/EP02/08094

§ 371 Date: Jul. 12, 2004

(30) Foreign Application Priority Data
Jul. 19, 2001 (EP) 01 11 6756.6

(51) Int. Cl. C40B 40/08; C40B 40/10; C12Q 1/68; G01N 33/53; C07H 21/04; C12P 21/06; C07K 16/18

(52) U.S. Cl. 435/6; 435/7.1; 435/69.1; 435/320.1; 435/326; 530/387.1; 536/23.53

(57) ABSTRACT

The present invention relates to a method for the optimization of isolated human immunoglobulin variable heavy (V_{H}) and light (V_{L}) constructs.

The present application claims priority on EP 01 11 6756.6 filed on Jul. 19^{th}, 2001, which hereby is incorporated by reference in its entirety.

### BACKGROUND OF THE INVENTION

Because of their high degree of specificity and broad target range, antibodies have found numerous applications in a variety of settings in basic research, clinical and industrial use, where they serve as tools to selectively recognize virtually any kind of substrate. However, despite their versatility there are intrinsic limitations in the use of antibody molecules for some important applications. For example, therapeutic or in vivo diagnostic antibody fragments require a long serum half-life in human patients to accumulate at the desired target, and they must, therefore, be resistant to precipitation and degradation by proteases (Willuda et al., 1999). Industrial applications often demand antibodies, that can function in organic solvents, surfactants or at high temperatures—all of which pose severe challenges to the stability of these molecules (Dooley et al., 1998; Harris et al., 1994). There is also a size consideration, especially in clinical applications. Enhanced tumor penetration favors smaller molecules, thus making the large size of whole antibodies a potential liability in some treatment regimens. Furthermore, the high demand for, and the increasing number of, applications of antibodies require more efficient methods for their high-level production.

Single-chain Fv (scFv) fragments are one antibody format designed to circumvent some of these limitations (Bird et al., 1988; Huston et al., 1988). The size of these molecules is reduced to the antigen binding part of an antibody, and they contain the variable domains of the heavy and light chain connected via a flexible linker. Most scFv fragments can be easily obtained from recombinant expression in E. coli in sufficient amounts (Glockshuber et al., 1992; Plückthun et al., 1996). As production yields of these fragments are influenced by their stability, as well as solubility and folding efficiency, considerable efforts have been made to identify positions in scFv fragments critical for influencing their expression behavior (Knappik & Plückthun, 1995; Forsberg et al., 1997; Kipriyanov et al., 1997; Nieba et al., 1997).

The factors influencing the stability of antibody molecules have been studied mostly with scFv fragments (Wörn & Plückthun, 2001). The overall stability of scFv fragments depends on the intrinsic structural stability of V_{L} and V_{H} as well as on the extrinsic stabilization provided by their interaction (Wörn & Plückthun, 1999). For some scFvs, the stabilities of isolated V_{H} and V_{L} domains, as well as of the whole scFv fragment, have been measured and compared recently (Jäger et al., 2001; Jäger & Plückthun, 1999a; Wörn & Plückthun, 1999). The V_{H} domain of the anti-HER2 scFv hu4D5-8, which was generated by loop grafting on a human V_{H}3 consensus framework (Carter et al., 1992; Rodrigues et al., 1992), shows a free energy of unfolding of 14.4 kJ/mol⁻¹ l (Jäger et al., 2001). This low thermodynamic stability is surprising at first glance, but there are several differences in framework residues of the V_{H}3 consensus sequence introduced after the loop grafting to increase affinity to HER2 (Carter et al., 1992). The V_{H} domain IcaH-01 of a catalytic antibody (Ohage et al., 1999) was engineered for stability by converting it to the consensus sequence (Steipe et al., 1994). Because of the frequent usage of V_{H}3 domains, this overall consensus is heavily biased towards the V_{H}3 consensus. Seven positions were identified and separately exchanged (Wirtz & Steipe, 1999).

ScFv fragments, as well as complete human antibodies against a broad variety of tailored antigens, can now be obtained from several antibody libraries (Griffiths et al., 1994; Vaughan et al., 1996; Knappik et al., 2000). The libraries are enriched by panning for antibody fragments that bind the desired target molecule, but the selection procedure is biased for additional factors such as expression behavior, toxicity of the expressed antibody construct to the bacterial host, protease sensitivity, folding efficiency, and stability. There are two conceivable solutions to make a diverse library of stable frameworks. The first is to use a single stable framework (Holt et al., 2000; Pini et al., 1998; Söderlind et al., 2000). These libraries use the germ line gene DP47 (Tomlinson et al., 1992) as the master framework for the V_{H} domain, since this gene is well expressed in bacterial systems (Griffiths et al., 1994) and most frequently expressed in vivo in human individuals (de Wildt et al., 1999). The Griffiths library is built from a germline V_{H} bank using in vitro generated CDR3 and FR4 sequences (Griffiths et al., 1994). The diversity has been reached by introducing various point mutations in the CDRs (Holt et al., 2000; Pini et al., 1998) or sampled CDRs from in vivo-processed gene sequences (Söderlind et al., 2000).

The second possibility to achieve a structurally diverse library of stable frameworks is to optimize the human consensus antibody frameworks further. Different frameworks with conformational changes for framework 1 conformations (Honegger & Plückthun, 2001 a; Jung et al., 2001; Saul & Poljak, 1993) may access a different range of CDR2 conformations (Saul & Poljak, 1993), while different framework 4 sequences affect CDR3 conformation. The Human Combinatorial Antibody Library (HuCAL, Knappik et al., 2000) consists of combinations of seven V_{H} and seven V_{L} synthetic consensus frameworks connected via a linker region forming 49 master genes (Knappik et al., 2000).

The basis for this library is a set of consensus sequences of the framework regions of the major V_{H}- and V_{L}-subfamilies (V_{H}1, V_{H}2, V_{H}3, V_{H}4, V_{H}5, and V_{H}6, Vκ1, Vκ2, Vκ3, Vκ4, Vλ1, Vλ2 and Vλ3). These subfamilies were identified from known germline sequences (VBASE, Cook & Tomlinson, 1995) with the V_{H}1 subfamily further divided into V_{H}1a and V_{H}1b because of different CDR-H2 conformations. For each of the subfamilies, a consensus sequence for the framework regions was calculated from a database of all known rearranged antibody sequences belonging to that subfamily.

These 14 consensus sequences ideally represent the structural repertoire of human variable domain frameworks.

These consensus sequences containing germline CDR1 and CDR2 sequences of the corresponding germline variable domain and identical CDR3s were used for expression studies (Knappik et al., 2000). Thus, it could be shown that the individual VH and VL domains are well expressed and stable in E. coli. However, these studies, and studies on their individual perfomance in recombinant libraries (Hanes et al., 2000) showed that nevertheless there are striking differences between the individual variable domains when compared to each other.

Enhanced overall expression and stability of antibodies or fragments thereof is highly desirable for most applications of antibody libraries.

Thus, the technical problem of the present invention is to improve the relative stability, overall expression and solubility of antibodies or fragments thereof. The solution to the above mentioned technical problem is achieved by providing the embodiments characterized in the claims and disclosed hereinafter.

The technical approach of the present invention i.e. modifying one or more framework residues in a human variable heavy or light chain antibody domain of a particular subclass with reference to a V_{H} or a V_{L} domain, respectively, of another subclass, is neither provided nor suggested by the prior art.

### SUMMARY OF THE INVENTION

The present invention provides antibodies having, inter alia, a modified framework region, using methods described and contemplated herein. Methods for mutating nucleic acid sequences are well known to the practitioner skilled in the art, including but not limited to cassette mutagenesis, site-directed mutagenesis, mutagenesis by PCR (see for example Sambrook et al., 1989; Ausubel et al., 1999).

In one aspect, the present invention provides isolated polypeptides (and isolated nucleic acid sequences encoding the same) that contain a V_{H} domain selected from the group consisting of (i) a V_{H} domain belonging to the V_{H}1a subclass, wherein the V_{H} domain contains an amino acid residue F at position 29 and/or L at position 89; (ii) a V_{H} domain belonging to the V_{H}1b subclass, wherein the V_{H} domain contains the amino acid residue L at position 89; (iii) a V_{H} domain belonging to the V_{H}2 subclass, wherein the V_{H} domain contains at least one amino acid residue selected from the group consisting of G at position 16, V at position 44, A at position 47, G at position 76, F at position 78, Y at position 90, R at position 97, E at position 99, wherein if R is at position 97, then E is at position 99; (iv) a V_{H} domain belonging to the V_{H}4 subclass, wherein the V_{H} domain contains at least one amino acid residue selected from the group consisting of G at position 16, A at position 47, F at position 78, Y at position 90, R at position 97, and E at position 99, wherein if R is at position 97, then E is at position 99; (v) a V_{H} domain belonging to the V_{H}5 subclass, wherein the V_{H} domain contains at least one amino acid residue selected from the group consisting of L at position 89, R at position 97, and E at position 99, wherein if R is at position 97, then E is at position 99; and (vi) a V_{H} domain belonging to the V_{H}6 subclass, wherein the V_{H} domain contains at least one amino acid residue selected from the group consisting of V at position 5, G at position 16, I at position 58, F at position 78, Y at position 90 and R at position 97, and E at position 99, wherein if R is at position 97, then E is at position 99.

The present invention also provides isolated polypeptides (and isolated nucleic acid sequences encoding the same) that contain a V_{L} domain selected from the group consisting of (i) a V_{L} domain belonging to the V_{L}κ2 subclass, wherein the V_{L} domain contains the amino acid residue R at position 18, and wherein if R is at position 18, then T is at position 92; and (ii) a V_{L} domain belonging to the V_{L}λ1 subclass, wherein the V_{L} domain contains the amino acid residue K at position 47.

The nucleic acid sequences encoding the polypeptides of the invention can be used, e.g., for the construction of libraries of antibodies or fragments thereof. Libraries of antibodies or fragments thereof have been described in various publications (see, e.g., Vaughan et al., 1996; Knappik et al., 2000; U.S. Pat. No. 6,300,064, which are incorporated by reference in their entirety), and are well-known to one of ordinary skill in the art.

In the context of the present invention, the term “V_{H} domain” refers to the variable part of the heavy chain of an immunoglobulin molecule. The term “V_{H} . . . subclass” includes the subclass defined by the corresponding “V_{H} . . . ” consensus sequence taken from the HuCAL (V_{H}1a, V_{H}1b, V_{H}2, V_{H}3, V_{H}4, V_{H}5, and V_{H}6 (Knappik et al., 2000) generated as described above. In this context, the term “subclass” refers to a group of variable domains sharing a high degree of identity and similarity represented by a consensus sequence of the major V_{H}-subfamilies, wherein the term “subfamily” is used as a synonym for “subclass.” In the context of the present invention, the term “consensus sequence” refers to the HuCAL consensus genes. The determination whether a given V_{H} domain is “belonging to a V_{H} subclass” is made by alignment of the V_{H} domain with all known human V_{H} germline segments (VBASE, Cook & Tomlinson, 1995) and determination of the highest degree of homology using a homology search matrix such as BLOSUM (Henikoff & Henikoff, 1992). Methods for determining homologies and grouping of sequences according to homologies are well known to one of ordinary skill in the art. The grouping of the individual germline sequences into subclasses is done according to Knappik et al., (2000).

In the context of the present invention the term “V_{L} domain” refers to the variable part of the light chain of an immunoglobulin molecule. The term “V_{L} . . . subclass” refers to the subclass defined by the corresponding V_{L} . . . consensus sequence taken from the HuCAL (Vκ1, Vκ2, Vκ3 and Vκ4 as well as Vλ1, Vλ2 and Vλ3; Knappik et al., 2000) generated as described above.

In this library, a consensus sequence for each of the major V_{L}-subfamilies was generated from known antibody sequences (VBASE, Cook & Tomlinson, 1995). In the context of the present invention, the numbering of the amino acid residues is according to the structurally adjusted scheme of Honegger & Plückthun (2001b).

In the context or the present invention, the term “antibody” is used as a synonym for “immunoglobulin”. Antibodies or fragments thereof according to the present invention may be Fv (Skerra & Plückthun, 1988), scFv (Bird et al., 1988; Huston et al., 1988), disulfide-linked Fv (Glockshuber et al., 1992; Brinkmann et al., 1993), Fab, (Fab′)₂ fragments, single V_{H} domains or other fragments well-known to the practitioner skilled in the art, which comprise at least one variable domain of an immunoglobulin or immunoglobulin fragment and have the ability to bind to a target.

### DETAILED DESCRIPTION

The invention provides novel immunoglobulin sequences and methods for making the same. The present inventors surprisingly discovered a scheme for optimizing certain framework regions of an immunoglobulin of any variable heavy or light chain subclass, using the sequences of another subclass (i.e., subfamily) as a reference point. The present invention, also relates to a method for the further modification of such optimized human variable domains comprising the steps of: (i) identifying for said domain the corresponding amino acid consensus sequence selected from the group of VH consensus sequences consisting of VH1a, VH1b, VH2, VH4, VH5, and VH6, and (ii) substituting one or more codons corresponding to amino acid residues of said consensus sequence into a corresponding position(s) in said nucleic acid sequence of said domain.

The following procedure describes a generally applicable method for improving the properties of any given human immunoglobulin heavy chain variable domain while keeping binding activity. (This method can be readily modified, using the guidance provided herein, to improve the properties of any given human immunoglobulin light chain variable domain). The first task is to compare each residue of the given domain to different subsets of immunoglobulin sequences. As the binding activity preferably is retained, residues of CDR1 (25-40), CDR2 (57-77), CDR3 (109-137) and the outer loop (84-87) are generally not considered (numbering scheme according to Honegger and Plückthun (2001b)). After determination of the framework 1 class, the subtype-determining (6, 7, 9, 10) and subtype-corresponding (19, 74, 78, 93) residues are compared to the consensus of sequences falling into the same class (Honegger and Plückthun, 2001a). The other residues are then compared to the consensus sequences of the V_{H} domains with favorable properties (families 1, 3 and 5) (see Example 1, Knappik et al., 2000). Next, the differences in residues are analyzed using structure models (see Example 2). Mutations that increase the expression yield of soluble protein and/or thermodynamic stability, as seen in this study, include: (i) mutations which replace a non-glycine residue in a loop with a positive phi-angle to glycine, (ii) mutations of residues in a β-strand with low β-sheet propensity to a residue with high β-sheet propensity, (iii) mutations of solvent exposed hydrophobic residues to hydrophilic ones, and (iv) replacement of residues with unsatisfied H-bonds.

In a preferred embodiment, the present invention relates to a method for the modification of certain human V_{H} domains belonging to a V_{H} subclass which is not V_{H}3, comprising the steps of: (a) identifying certain amino acid residues of said V_{H} domain being different compared to the corresponding amino acid residues of the HuCAL V_{H}3 domain, (b) replacing at least one of the differing amino acid residues by the corresponding amino acid residues of the HuCAL V_{H}3 domain, provided that the replacing amino acid residue is not the consensus amino acid residue of said subclass.

This basic method is, in principle, also applicable to V_{L} domains. For example, V_{κ} domains can be compared to the consensus sequence of V_{κ}3, as this domain displays the highest thermodynamic stability and expression yield of V_{κ} domains. The physical principles for rational design V_{λ} domains are the same as with V_{H} domains described above.

In a preferred embodiment, the present invention relates to an isolated polypeptide comprising a V_{H} domain belonging to the V_{H}1a subclass, wherein said V_{H} domain comprises an amino acid residue F at position 29 and L at position 89.

In yet a further embodiment, the invention relates to an isolated polypeptide comprising a V_{H} domain belonging to the V_{H}1b subclass, wherein said V_{H} domain comprises the amino acid residue L at position 89.

In a further preferred embodiment, the invention relates to an isolated polypeptide comprising a V_{H} domain belonging to the V_{H}2 subclass, wherein said V_{H} domain comprises at least one amino acid residue selected from the group consisting of G at position 16, V at position 44, A at position 47, G at position 76, F at position 78, Y at position 90, R at position 97, E at position 99, wherein if R is at position 97, then E is at position 99.

In yet a further preferred embodiment, the invention relates to an isolated polypeptide comprising a V_{H} domain belonging to the V_{H}4 subclass, wherein said V_{H} domain comprises at least one amino acid residue selected from the group consisting of G at position 16, A at position 47, F at position 78, Y at position 90, R at position 97, and E at position 99, wherein if R is at position 97, then E is at position 99.

In yet a further preferred embodiment, the invention relates to an isolated polypeptide comprising a V_{H} domain belonging to the V_{H}5 subclass, wherein said V_{H} domain comprises at least one amino acid residue selected from the group consisting of L at position 89, R at position 97, and E at position 99, wherein if R is at position 97, then E is at position 99.

In a further preferred embodiment, the present invention relates to an isolated polypeptide comprising a V_{H} domain belonging to the V_{H}6 subclass, wherein said V_{H} domain comprises at least one amino acid residue selected from the group consisting of V at position 5, G at position 16, I at position 58, F at position 78, Y at position 90 and R at position 97, and at position 99, wherein if R is at position 97, then E is at position 99.

In yet a further preferred embodiment, the invention relates to an antibody or functional fragment thereof comprising any V_{H} domain according to the present invention. Further preferred is a library of antibodies or functional fragments thereof comprising one or more antibodies or functional fragments thereof according to the present invention.

A library according to the present invention could be generated, starting from the HuCAL library (Knappik et al., 2000) by optimizing one or more of the VH and/or VL consensus sequences in accordance with the teaching of the present invention, and by introducing diversity into at least one CDR region in said optimized sequence, e.g. by using oligonucleotide cassettes synthesized using trinucleotide-directed mutagenesis as described in Knappik et al., 2000.

In yet a further preferred embodiment, the present invention relates to an isolated polypeptide comprising a V_{L} domain belonging to the V_{L}κ2 subclass, wherein said V_{L} domain comprises the amino acid residue R at position 18, and wherein R is at position 18, then T is at position 92.

In a further preferred embodiment, the present invention relates to an isolated polypeptide comprising a V_{L} domain belonging to the V_{L}λ1 subclass, wherein said V_{L} domain comprises the amino acid residue K at position 47.

In yet a further preferred embodiment, the present invention relates to an antibody or a functional fragment thereof comprising a V_{L} domain according to the present invention.

In a most preferred embodiment, the present invention relates to libraries of antibodies or functional fragments thereof comprising one or more antibodies or functional fragments thereof according to the present invention.

In a further preferred embodiment, the present invention relates to a method for the modification of a human V_{H} domain belonging to the V_{H}1a subclass by generating a modified V_{H} domain comprising at least one amino acid residue exchange taken from the list of: (a) 29 to F and (b) 89 to L.

In yet a further embodiment, the invention provides for a method for the modification of a human V_{H} domain belonging to the V_{H}1b subclass by generating a modified V_{H} domain comprising the amino acid residue exchange: 89 to L.

In a further embodiment, the invention relates to a method for the modification of a human V_{H} domain belonging to the V_{H}2 subclass by generating a modified V_{H} domain comprising at least one amino acid residue exchange taken from the list of: (a) 16 to G; (b) 44 to V; (c) 47 to A; (d) 76 to G; (e) 78 to F; (f) 97 to R, provided that the amino acid residue 99 is, or is exchanged to E; and (g) 99 to E. Further preferred is a method for the modification of a V_{H} domain belonging to the V_{H}2 subclass, by generating a modified V_{H} domain comprising the amino acid residue exchange 90 to Y.

In a further preferred embodiment, the invention relates to a method for the modification of a human V_{H} domain belonging to the V_{H}4 subclass by generating a modified V_{H} domain comprising at least one amino acid residue exchange taken from the list of: (a) 16 to G; (b) 44 to V; (c) 47 to A; (d) 76 to G; (e) 78 to F; (f) 97 to R, provided that the amino acid residue 99 is, or is exchanged to E; and (g) 99 to E. Further preferred is a method for the modification of a human V_{H} domain belonging to the V_{H}4 subclass, by generating a modified V_{H} domain comprising the amino acid residue exchange 90 to Y.

In a further preferred embodiment, the invention provides for a method for the modification of a human V_{H} domain belonging to the V_{H}5 subclass by generating a modified V_{H} domain comprising at least one amino acid residue exchange taken from the list of: (a) 77 to R; (b) 89 to L; (c) 97 to R, provided that the amino acid residue 99 is, or is exchanged to E; and (d) 99 to E.

In yet a further embodiment, the invention provides for a method for the modification of a human V_{H} domain belonging to the V_{H}6 subclass by generating a modified V_{H} domain comprising at least one amino acid residue exchange taken from the list of: (a) 5 to V; (b) 16 to G; (c) 44 to V; (d) 58 to I; (e) 72 to D; (f) 76 to G; (g) 78 to F and (h) 97 to R, provided that the amino acid residue 99 is, or is exchanged to E. Further preferred is a method for the modification of a V_{H} domain belonging to the V_{H}6 subclass, by generating a modified V_{H} domain comprising the amino acid residue exchange 90 to Y.

In another embodiment, the present invention relates to a method for the modification of a V_{H} domain, wherein 2 or more amino acid residues are exchanged.

In a further embodiment, the present invention provides for a method for the modification of a V_{H} domain comprising the steps of (i) providing a nucleic acid molecule encoding said V_{H} domain; (ii) mutating said nucleic acid molecule resulting in a modified nucleic acid molecule encoding said modified V_{H} domain.

In a preferred embodiment, the present invention relates to a method for obtaining a polypeptide according to the present invention, substituting in a V_{H}1a subclass domain at least one amino acid residue selected from the group consisting of F at position 29 and L at position 89.

In yet a further preferred embodiment, the present invention relates to a method for obtaining a polypeptide according to the present invention, comprising the step of substituting in a V_{H}1b subclass domain the amino acid residue L at position 89.

In a further preferred embodiment, the present invention relates to a method for obtaining a polypeptide according to the present invention, comprising the step of substituting in a V_{H}2 subclass domain at least one amino acid residue selected from the group consisting of G at position 16, V at position 44, A at position 47, G at position 76, F at position 78, R at position 97, and E at position 99, wherein if R is at position 97, then E is at position 99. Further preferred is a method for obtaining the polypeptide according to the present invention, comprising the step of substituting in a V_{H}2 subclass domain the amino acid residue Y at position 90.

In a further preferred embodiment, the present invention relates to a method for obtaining the polypeptide according to the present invention, comprising the step of substituting in a V_{H}4 subclass domain at least one amino acid residue selected from the group consisting of G at position 16, V at position 44, A at position 47, G at position 76, F at position 78, R at position 97, and E at position 99, wherein if R is at position 97, then E is at position 99. Further preferred is a method for obtaining the polypeptide according to the present invention, comprising the step of substituting in a V_{H}4 subclass domain the amino acid residue Y at position 90.

In yet a further preferred embodiment, the present invention relates to a method for obtaining the polypeptide according to the present invention, comprising the step of substituting in a V_{H}5 subclass domain at least one amino acid residue selected from the group consisting of R at position 77, L at position 89, R at position 97, and E at position 99, wherein if R is at position 97, then E is at position 99.

In a further preferred embodiment, the present invention relates to a method for obtaining a polypeptide according to the present invention, comprising the step of substituting in a V_{H}6 subclass domain at least one amino acid residue selected from the group consisting of V at position 5, G at position 16, V at position 44, I at position 58, D at position 72, G at position 76, F at position 78,R at position 97, and E is at position 99, wherein if R is at position 97, then E is at position 99. Further preferred is a method for obtaining a polypeptide according to the present invention, comprising the step of substituting in a VH6 subclass domain the amino acid residue Y at position 90.

In a further preferred embodiment, the present invention relates to a method for obtaining a polypeptide according to the present invention, wherein 2 or more amino acid residues are substituted.

In yet a further preferred embodiment, the present invention relates to a method for obtaining the polypeptide according to the present invention, comprising the step of substituting in a of a V_{L}κ2 subclass domain at least one amino acid residue selected from the group consisting of S at position 12, Q at position 45, and R at position 18, and wherein R is at position 18, then T is at position 92.

In yet a further preferred embodiment, the present invention relates to a method for obtaining the polypeptide according to the present invention, comprising the step of substituting in a V_{L}λ1 subclass domain at least one amino acid residue selected from the group consisting of K at position 47.

In a further preferred embodiment, the present invention relates to a method for obtaining a polypeptide according to the present invention, comprising the step of substituting in a V_{L}λ1, V_{L}λ2 and V_{L}λ3 domain the amino acid residue P at position 8. Further preferred is a method for obtaining a polypeptide according to the present invention, wherein P is at position 8, and further comprising the substitutions S at positions 7 and 9.

In a further preferred embodiment, the present invention relates to a method according to the present invention, wherein 2 or more amino acid residues are substituted.

In a further preferred embodiment, the present invention relates to a method for obtaining a polypeptide according to the present invention further comprising the step of expressing a modified nucleic acid molecule.

In a further preferred embodiment, the present invention relates to an isolated nucleic acid molecule encoding an inventive V_{H} domain, an antibody or a functional fragment thereof, as disclosed or contemplated herein.

In a further preferred embodiment, the present invention relates to an isolated nucleic acid molecule encoding an inventive V_{L} domain, an antibody or a functional fragment thereof, as disclosed or contemplated herein.

In a further preferred embodiment, the present invention relates to a method for producing a V_{L} domain, antibody or a functional fragment thereof, as described or contemplated herein, comprising the step of expressing an isolated nucleic acid molecule of the present invention.

The invention also provides for conservative amino acid variants of the molecules of the invention. Variants according to the invention also may be made that conserve the overall molecular structure of the encoded proteins. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

For example: (a) nonpolar (hydrophobic) amino acids include alanine, leucine, isoleucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in αhelices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and 1. Given the known genetic code, and recombinant and synthetic DNA techniques, the skilled scientist readily can construct DNAs encoding the conservative amino acid variants.

As used herein, “sequence identity” between two polypeptide sequences indicates the percentage of amino acids that are identical between the sequences. “Sequence similarity” indicates the percentage of amino acids that either are identical or that represent conservative amino acid substitutions.

The invention also provides nucleic acids that hybridize under high stringency conditions to the V_{H} and/or V_{L} domains, antibodies or functional fragments thereof, according to the present invention. As used herein, highly stringent conditions are those, which are tolerant of up to about 5-20% sequence divergence, preferably about 5-10%. Without limitation, examples of highly stringent (−10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5×Denhardt's reagent, 0.5% SDS, 100 μg/ml denatured, fragmented salmon sperm DNA at an appropriate incubation temperature Ti. See generally Sambrook et al., Molecular Cloning: A Laboratory Manual, 2d edition, Cold Spring Harbor Press (1989)) for suitable high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na+. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as
Tm=81.5° C.+16.6(log M)+0.41(% GC)−0.61(% form)−500/L
and for DNA:RNA hybrids, as
Tm=79.8° C.+18.5(log M)+0.58(% GC)−11.8(% GC)2−0.56(% form)−820/L
where M, molarity of monovalent cations, 0.01-0.4 M NaCl,
% GC, percentage of G and C nucleotides in DNA, 30%-75%,
% form, percentage formamide in hybridization solution, and
L, length hybrid in base pairs.

Tm is reduced by 0.5-1.5° C. (an average of 1° C. can be used for ease of calculation) for each 1% mismatching.

The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

In a further preferred embodiment, the present invention relates to a method for producing a V_{H} domain, antibody or a functional fragment thereof, as described or contemplated herein, comprising the step of expressing an isolated nucleic acid molecule of the present invention.

In particular, such method comprises the steps of: (i) providing a nucleic acid molecule encoding a V_{H} domain; (ii) mutating said nucleic acid molecule resulting in a modified nucleic acid molecule encoding a modified V_{H} domain comprising at least one amino acid residue exchange. Methods for mutating nucleic acid sequences are well known to the practitioner skilled in the art, encluding but not limited to cassette mutagenesis, site-directed mutagenesis, mutagenesis by PCR (see for example Sambrook et al., 1989; Ausubel et al., 1999).

Further preferred is a vector comprising an isolated nucleic acid molecule according to the present invention.

In yet a further preferred embodiment, the invention relates to a host cell harboring an isolated nucleic acid molecule according to the present invention or a vector according to the present invention.

In a further preferred embodiment, the V_{H} domains according to the present invention can be used for all applications of antibodies including but not limited to the construction, generation, expression and screening of antibody libraries.

In a further preferred embodiment, the V_{L} domains according to the present invention can be used for all applications of antibodies including but not limited to the construction, generation, expression and screening of antibody libraries

In yet a further preferred embodiment, the present invention relates to an antibody or a functional fragment thereof (and methods of making the same), that contains any combination of a V_{H} and V_{L} domain described herein. For example, an antibody may comprise (i) a V_{H} domain belonging to the V_{H}1a subclass, wherein said V_{H} domain comprises an amino acid residue F at position 29 and/or L at position 89; and (ii) a V_{L} domain belonging to the V_{L}κ2 subclass, wherein said V_{L} domain comprises one or more of the following substitutions: S at position 12, Q at position 45, or R at position 18, provided that if R is at position 18, then T is at position. 92.

In still a further preferred embodiment, the present invention relates to a library of antibodies or functional fragments thereof comprising one or more antibodies or functional fragments thereof, according to the present invention.

In a further preferred embodiment, the present invention relates to an isolated nucleic acid molecule encoding an antibody or functional fragment thereof according to the present invention.

### FIGURE CAPTIONS

FIG. 1. Determination of apparent molecular mass of isolated V_{H} and V_{L} domains. Gel filtration runs were performed in 50 mM sodium-phosphate (pH 7.0) and 500 mM NaCl of (a) isolated human consensus V_{H} domains (5 μM) on a Superdex-75 column with V_{H}3 (solid line) and V_{H}1a (dotted line) and V_{H}1a in the presence of 0.9 M GdnHCl (long dashed line); (b) isolated V_{κ} domains (50 μM) on a Superose-12 column with V_{κ}1 (solid), V_{κ}2 (long dashed), V_{κ}3 (dotted) and V_{κ}4 (short dashed line); and (c) isolated V_{λ} domains (5 μM) on a TSK column with V_{λ}1 (solid), V_{λ}2 (long dashed) and V_{λ}3 (dotted line). Arrows indicate elution volumes of molecular mass standards: carbonic anhydrase (29 kDa), and cytochrome c (12.4 kDa). (d) Equilibrium sedimentation of V_{κ}3 at 19,000 rpm with a detection wavelength of 280 nm. The solid line was obtained from fitting of the data to a single species, and a molecular weight of 13616 Da was calculated. The residuals of the fit are scattered randomly, indicating that the assumption of the monomeric state is valid.

FIG. 2. Overlay of GdnHCl denaturation curves of V_{H} domains (a) V_{H}1a (filled circles), V_{H}1b (open squares), V_{H}3 (filled squares) and V_{H}5 (open circles). (b) V_{H}2 (filled circles), V_{H}4 (open squares) and V_{H}6 (filled squares). All unfolding transitions (a and b) were measured by following the change in emission maximum as a function of denaturant concentration at an excitation wavelength of 280 nm.

FIG. 3. Overlay of GdnHCl denaturation curves of V_{L} domains (a) V_{κ} domains with V_{κ}1 (filled circles), V_{κ}2 (filled squares), V_{κ}3 (open squares) and V_{κ}4 (open circles) and (b) V_{λ} domains with V_{λ}1 (filled squares), V_{λ}2 (filled circles), V_{λ}3 (open squares). All unfolding transitions (a and b) were measured by following the change of fluorescence intensity as a function of denaturant concentration at an excitation wavelength of 280 nm.

FIG. 4. Model structure of a scFv fragment consisting of human consensus V_{κ}3 (PDB entry: 1DH5) and V_{H}3 domain (PDB entry: 1DHU). (a) Secondary structure with V_{κ}3 on the left and V_{H}3 on the right side (b) Marked for charged residues (grey: Arg, Lys and His; black: Asp and Glu). At the base of each domain is an accumulation of charged residues, the charge clusters of V_{L} and V_{H} domains. (c) Hydrophobic core residues: Above the conserved Trp43 (light grey) is the upper core (dark grey) and below the lower core (black), see text for details. (d) Positions possibly influencing folding efficiency are shown in light grey, see text for details. All images were generated using the program MOLMOL (Koradi et al., 1996).

FIG. 5. Detailed view of the charge cluster of the human consensus (a) V_{H}3 and (b) V_{κ}3 family with hydrogen bonds. Images were generated using the program MOLMOL (Koradi et al., 1996).

FIG. 6. Detailed view of the upper core residues. Superposition of (a) V_{H}4, (b) V_{H}1a and (c) V_{H}5, each in light grey, with V_{H}3 in black and (d) V_{λ}1 in light grey with V_{κ}3 in black, see text for details. The conserved Trp43 is shown. Residues 4, 80 and 82 are not shown, as they do not contribute to the packing differences discussed in the text. Images were generated using the program MOLMOL (Koradi et al., 1996).

FIG. 7. Detailed view of the lower core residues that correspond to framework 1 classification. Superposition of (Aa) V_{H}1a (light grey) and V_{H}3 (black) (Bb) V_{H}4 (light grey) and V_{H}3 (black) and (c) V_{λ}1 (light grey) and V_{κ}3 (black), see text for details. The conserved Trp43 is shown. Images were generated using the program MOLMOL (Koradi et al., 1996).

FIG. 8. Analytical gel filtration of scFv fragments (5 μM) on a Superdex-75 column in 50 mM sodium-phosphate (pH 7.0) and 500 mM NaCl: (a) H3κ3 (solid line), H4κ3 (long-dashed line), H1aκ3 (short-dashed line) and H1aκ3 in the presence of 1 M GdnHCl (short-dashed line). (b) H3κ3 (solid line), H3κ1 (long-dashed line), H3λ1 (short-dashed line) and H3λ1 in the presence of 1 M GdnHCl (short-dashed line). Arrows indicate elution volumes of molecular mass standards: bovine serum albumin (66 kDa), carbonic anhydrase (29 kDa), and cytochrome c (14 kDa).

FIG. 9. Overlay of GdnHCl denaturation curves to illustrate different cases of interface stabilization. In each panel the scFv fragment (filled squares) and accompanying isolated V_{H} (open squares) and V_{L} (open circles) domains are shown. All unfolding transitions in (a) with H5κ3, (b) with H1aκ3, (c) with H3κ1 and (d) with H3κ2 were measured by following the change in emission maximum (in case of scFv fragments and V_{H} domains) or fluorescence intensity (in case of V_{L} domains) as a function of denaturant concentration at an excitation wavelength of 280 nm.

FIG. 10. Overlay of GdnHCl denaturation curves to illustrate the role of different L-CDR3 in interface stabilization in V_{λ} domains. In (a) with H3λ1 with the λ-like L-CDR3 and (b) with H3λ1 with the κ-like L-CDR3 the scFv fragments (filled squares) and constituent isolated V_{H}3 (open squares) and V_{λ}1 (open circles) domains are shown. As the isolated V_{λ} domains with the κ-like CDR3 show non-reversible behavior, in (b) the renaturation curve of V_{λ}1 is also shown (filled circles). All unfolding transitions were measured by following the change in emission maximum (in case of scFv fragments and V_{H} domains) or fluorescence intensity (in case of V_{L} domains) as a function of denaturant concentration at an excitation wavelength of 280 nm.

FIG. 11. Analytical gel filtration of 2C2-wt, 2C2-all, 6B3-wt and 6B3-all in 50 mM sodium-phosphate (pH 7.0) and 500 mM NaCl on a Superdex-75 column at a concentration of 5 μM. 6B3-wt (long-dashed line) and 6B3-all (dotted line) show a similar elution volume. Arrows indicate elution volumes of molecular mass standards: bovine serum albumin (66 kDa), carbonic anhydrase (29 kDa), and cytochrome c (12.4 kDa). The mutations carried by 2C2-all and 6B3-all are listed in Table 7 and FIG. 12.

FIG. 12. Overlay of GdnHCl denaturation curves of (a) of 2C2-wt, 2C2-all, 6B3-wt and 6B3-all, (b) single mutations (abbreviations used: a=Q5V, b=S16G, c=T58I, d=V72D, e=S76G, f=S90Y and all=abcdef) and (c) multiple mutations to the consensus of V_{H} domains with favorable properties and (d) mutations (abbreviations used: g=P10A and gh=P10A+V74F) to the framework 1 subtype III exemplified with the scFv 2C2. In (b), (c) and (d) the bold solid line and the bold dotted line represent the fits (Jäger et al., 2001) of the experimental data shown in (a) of 2C2-wt and 2C2-all, respectively. All unfolding transitions were measured by following the change in emission maximum as a function of denaturant concentration at an excitation wavelength of 280 nm.

FIG. 13. Aligned HuCAL V_{H} sequences. The amino acids are shaded according to residue type: aromatic residues (Tyr, Phe, Trp), hydrophobic residues (Leu, Ile, Val, Met, Cys, Pro, Ala), uncharged hydrophilic residues (Ser, Thr, Gln, Asn, Gly), acidic residues (Asp, Glu), basic residues (Arg, Lys; His). Residues that show correlated sequence differences between the groups of V_{H} domains with favorable properties (V_{H}1a, V_{H}1b, V_{H}3, V_{H}5) and V_{H} domains with less favorable properties (V_{H}2, V_{H}4, V_{H}6) indicated by white boxes. Numbering scheme is according to Kabat et al. (1991) and Honegger & Plückthun (2001b).

FIG. 14. Overview of the single mutations to the consensus of those V_{H} domains with favorable properties. In the middle of the figure a model scFv fragment consisting of V_{H}6 (black ribbon, PDB entry: 1DHZ) and V_{L}κ3 domain (gray ribbon, PDB entry: 1DH5) is shown with the single mutations indicated by arrows, that point to enlargements of the single mutations. All images were generated using the program MOLMOL (Koradi et al., 1996). Numbering scheme is according to Honegger & Plückthun (2001b).

FIG. 15. Overview of framework 1 subtype III determining residues (6, 7 and 10) and correlated residues (19, 74, 78, 93) (a) in the wild type V_{H}6 domain (PDB entry: 1DHZ) and (b) in the model of the double mutated form with the changes P10A and V74F. (c) Ribbon representation of the V_{H}6 domain with black frame indicating the enlarged area depicted in (a) and (b). All images were generated using the program MOLMOL (Koradi et al., 1996). Numbering scheme according to Honegger & Plückthun (2001b).

FIG. 16. Comparison of the binding activities of (a) 2C2-wt and 2C2-all and (b) 6B3-wt and 6B3-all. BIAcore experiments are shown, with resonance units plotted against time after injection of different scFv concentrations over an antigen-coated chip. Solid lines indicate wild-type scFv fragments and dotted lines indicate scFv fragments carrying all six mutations toward the consensus of favorable V_{H} domains. In (a) 2C2-wt and 2C2-all at concentrations of 1.25, 0.63, 0.31 and 0.16 μM and in (b) 6B3-wt and 6B3-all at concentrations of 1.25, 0.63, 0.31, 0.16 and 0.08 μM are plotted.

FIG. 17. Competition BIAcore analysis of 6B3-wt and 6B3-all. (a) 6B3-wt (16 nM) and (b) 6B3-all (10 nM) were incubated with different concentrations of myoglobin for 1 hour and injected over a myoglobin-coated sensor chip. From the linear sensograms, the slopes (resonance units vs. time in sec) were plotted against the corresponding total soluble antigen concentration. The slopes correlate to uncomplexed scFv in the injected solutions. K_{d} was calculated from a fit according to Hanes et al (1998). Each point is the average of three independent measurements. The example illustrates the invention.

### EXAMPLES

In the following examples, all molecular biology experiments are performed according to standard protocols (Ausubel et al., 1999).

### Example 1

Construction of Expression Vectors

Starting point for all expression vectors were the scFv master genes of the HuCAL library in the orientation V_{H}-(Gly₄Ser)₄-V_{L} in the expression vector pBS13 (Knappik et al., 2000), which all carried H-CDR3 and L-CDR3 of the antibody hu4D5-8 (Carter et al., 1992).

The seven isolated human consensus V_{H} domains were PCR amplified from the master genes and the CDR3 region between the BssHII and StyI restriction sites was then exchanged to code for a CDR-H3 found by metabolic selection (J. Burmester et al., unpublished results): YNHEADMLIRNWLYSDV. The final expression plasmids were derivatives of the vector pAK400 (Krebber et al., 1997), in which the expression cassette of the seven different V_{H} domains had been introduced between the XbaI and HindIII restriction sites, and where the skp cassette (Bothmann & Plückthun, 1998) had been introduced at the NotI restriction site. The expression cassette consists of a phoA signal sequence, the short FLAG-tag (DYKD), one of the seven V_{H} domains and a hexahistidine-tag.

The seven isolated human consensus V_{L} domains were cut out from the master genes with the restriction enzymes EcoRV and EcoRI and ligated into a pAK400 derivative with these restriction sites. The L-CDR3 of the V_{λ} domains between the BbsI and MscI restriction sites was exchanged to QSYDSSLSGVV (107-138). This λ-like L-CDR3 is a consensus L-CDR3 from sequences found in the Kabat database (Kabat et al., 1991) for V_{λ} domains, in contrast to the κ-like L-CDR3 of hu-4D5-8 with the conserved cis-proline in position 136. The chosen length of the consensus λ-like L-CDR3 is found in 20% of the sequences, representing the highest percentage. The tryptophan at position 109, which is the most frequent residue with 54%, was exchanged to tyrosine, which is present in 20% of the sequences, to avoid interference with the native state fluorescence signal of the conserved unique tryptophan. The final expression cassette consists of a pelB signal sequence, one of the seven V_{L} domains and a hexahistidine-tag.

The scFv fragments were cloned via the restriction sites XbaI and EcoRI into the expression plasmid pMX7. The κ-like L-CDR3 was exchanged in the V_{λ} domains as reported above. The final expression cassette consists of a phoA signal sequence, the short FLAG-tag (DYKD), one of the seven V_{H} domains a (Gly₄Ser)₄ linker and one of the seven V_{L} domains, the long FLAG-tag (DYKDDDD) and a hexahistidine-tag.

Soluble Periplasmic Expression

dYT medium (30 ml containing 30 μg/mL chloramphenicol, 1.0% glucose) was inoculated with a single bacterial colony and incubated overnight at 25° C. One liter of dYT media (30 μg/mL chloramphenicol, 50 mM K₂HPO₄) was inoculated with the preculture and incubated at 25° C. (5 L flask with baffles, 105 rpm). Expression was induced at an OD₅₅₀ of 1.0 by addition of IPTG to a final concentration of 0.5 mM. Incubation was continued for 18 hours, when the cell density reached an OD₅₅₀ between 8.0 and 11.0. Cells were collected by centrifugation (8000 g, 10 minutes at 4° C.), suspended in 40 ml of 50 mM Tris-HCl (pH 7.5) and 500 mM NaCl and disrupted by French Press lysis. The crude extract was centrifuged (48,000 g, 60 minutes at 4° C.), the supernatant passed through a 0.2 μm filter and directly applied to IMAC chromatography.

Preparative Two-Column Purification

The proteins were purified using the two column coupled in-line procedure (Plückthun et al., 1996). In this strategy, the eluate of an immobilized metal ion affinity chromatography (IMAC) column, which exploits the C-terminal His-tag, was directly loaded onto an ion-exchange column. Elution from the ion-exchange column was achieved with a 0-800 mM NaCl gradient. The V_{H} and V_{κ} domains were purified with a HS cation-exchange column in 10 mM MES (pH 6.0) and the V_{λ} domains and the scFv fragments with an HQ anion-exchange column in 10 mM Tris-HCl (pH 8.0). Pooled fractions were dialyzed against 50 mM Na-phosphate, pH 7.0, 100 mM NaCl.

Insoluble Periplasmic Expression

LB medium (30 ml, containing 30 μg/ml chloramphenicol, 1% glucose) was inoculated with a single colony and incubated overnight at 37° C. One liter of SB medium (10 μg/ml chloramphenicol, 0.1% glucose, 0.4 M sucrose) was inoculated with 10 ml of the preculture and incubated at 25° C. Expression was induced at an OD₅₅₀ of 0.8 by addition of IPTG to a final concentration of 0.05 mM. Incubation was continued for about 15 hours at 25° C. After centrifugation, cells were suspended in 100 mM Tris-HCl, pH 8.0, 2 mM MgCl₂ and disrupted by French Press lysis. Inclusion bodies were isolated following a standard protocol (Buchner & Rudolph, 1991). The inclusion body pellet from 1 l bacterial culture was solubilized at room temperature in 10 ml of solubilization buffer (0.2 M Tris-HCl, pH 8.0, 6 M guanidine hydrochloride (GdnHCl), 10 mM EDTA, 50 mM DTT). The resulting solution was centrifuged and the supernatant dialyzed against solubilization buffer without DTT at 10° C. The sample was loaded on a nitrilotriacetic acid column (Qiagen), which had been charged with Ni²⁺, and IMAC under denaturating conditions was performed. The eluate was diluted (1:10) into refolding buffer (0.5 M Tris-HCl, pH 8.5, 0.4 M arginine, 5 mM EDTA, 20% glycerol, 0.5 mM ε-amino-caproic acid, 0.5 mM benzamidinium-HCl) at 16° C. at a final protein concentration of 1 μM. The formation of disulfide bonds was catalyzed either by the presence of reduced and oxidized glutathione in the refolding buffer at molar concentrations of [GSH]:[GSSG] 0.2:1 mM (oxidizing conditions) or 5:1 mM (reducing conditions). The refolding mixture was incubated at 16° C. for 20 hours and dialyzed against 50 mM Na-phosphate, pH 7.0, 100 mM NaCl.

Ni-NTA Batch Purification

Twenty mL of the supernatant of the French press lysis of the scFv fragments was incubated with 2 mL of a 50% Ni-NTA slurry for 30 min at room temperature. The suspension was applied on a empty column with a diameter of 1.5 cm and washed extensively with 50 mM sodium-phosphate (pH 7.0) and 1 M NaCl. To remove unspecific binding proteins, the column was washed with 30 mM imidazole. The scFv fragments were eluted by adding 250 mM imidazole. The purity of the samples was checked by SDS-PAGE analysis and the concentration was determined by absorbance at 280 nm. Four scFv fragments were purified in parallel with H3κ3 always as a control. The yield was normalized to the yield of H3κ3 and to a 1 L expression culture with an OD₅₅₀ of 10.

Determination of Insoluble Protein Ratio

An aliquot of a French press lysis extract of a 1 L scFv fragment expression experiment was centrifuged at 4° C. for 30 minutes at 16000 g. The supernatant (soluble fraction) and the precipitate (insoluble fraction), which was resuspended in 50 mM Tris-HCl (pH 7.5) and 500 mM NaCl, were analyzed by SDS-PAGE followed by Western Blot with the anti-His antibody 3D5 as described (Lindner et al., 1997). Chemiluminiscence was detected using a ChemiImager™ 4400 (Alpha Innotech Corporation) and the density of the bands were determined with the software ChemiImager™ 5500 (Alpha Innotech Corporation). As the method involves many steps, the error is possibly high, and therefore we give the values as a percentage of insoluble material, rounded to tens, with an estimated error of 10%.

Gel Filtration Chromatography

Samples of purified proteins were analyzed on a gel filtration column equilibrated with 50 mM Na-phosphate, pH 7.0, 500 mM NaCl. The isolated V_{H} domains and the scFv fragments at a concentration of 5 μM were injected on a Superdex-75 column (Pharmacia) and the isolated V_{κ} domains at a concentration of 50 and 5 μM on a Superose-12 column (Pharmacia) in a volume of 50 μL and a flow-rate of 60 μL/min on a SMART-system (Pharmacia). The V_{λ} domains were injected on a silica based TSK-Gel® G3000SWXL column (TosoH) on a HPLC system (HP) in a volume of 50 μL at a concentration of 5 μM and a flow rate of 0.5 mL/min. Lysozyme (14 kDa), carbonic anhydrase (29 kDa) and bovine serum albumin (66 kDa) were used as molecular standards. Elution was followed by detection of the absobance at 280 nm in the case of the SMART-system and at 220 nm in the case of the HPLC system.

Ultracentrifugation

Sedimentation equilibria were determined with a XL-A analytical ultracentrifuge (Beckmann). The samples were dialyzed against 10 mM sodium-phosphate (pH 7.0) and 100 mM NaCl overnight and loaded into a standard 6 channel 12 mm pathlength cell at a sample OD₂₈₀ of 0.4. The fluorocarbon FC43 was added to each cell sector to provide a false bottom. The samples were run for 24 h at 20° C. at 19000 rpm. Data were collected at 280 nm at a radial spacing of 0.001 cm and a minimum of 10 scans were averaged for each sample. Data were analyzed with software provided by the instrument manufacturer using models that assumed either the presence of a single species or of a monomer-dimer equilibrium as described previously (Liu et al., 1998). Solvent densities and sample partial volumes were calculated using standard methods.

Expression and Protein Purification of V_{H} Domains

The seven HuCAL consensus V_{H} domains representing the major framework subclasses were expressed with the same CDR-H3 to enable the comparison of their biophysical properties. First the V_{H} domains were investigated with the CDR3 from the antibody hu4D5-8 (WGGDGFYAMDY) (Carter et al., 1992), but the V_{H} domains were insoluble when expressed on its own, and only a small inclusion body pellet was obtained. This was not surprising, as many if not most V_{H} domains by themselves are insoluble upon periplasmic expression (Jäger et al., 2001; Jäger & Plückthun, 1999b; Wirtz & Steipe, 1999), since they contain an exposed large hydrophobic interface which is usually covered by V_{L}. However, recently three isolated V_{H} domains from the HuCAL (with framework classes V_{H}1a, V_{H}1b, and V_{H}3) have been selected in a metabolic selection experiment. These could be expressed in the periplasm of E. coli and purified from the soluble fraction of the cell extracts. The main feature of the selected V_{H} domains is the length of the CDR3, as all three selected and soluble V_{H} fragments contain a longer CDR3. This long CDR3 may cover the hydrophobic interface of V_{H}, thereby preventing aggregation. After introducing the CDR3 from one of the selected V_{H}3 domains (YNHEADMLIRNWLYSDV), V_{H}1a, V_{H}1b and V_{H}3 could be expressed in soluble form in the periplasm of E. coli and purified from the soluble fraction of the cell extracts with a yield of 2 mg/l.

In contrast, V_{H}2, V_{H}4, V_{H}5 and V_{H}6 were still insoluble in the E. coli periplasm. These domains were purified from the insoluble fraction with IMAC under denaturating conditions, and the eluted fractions were subjected to in vitro refolding. Approximately 1 mg soluble, refolded V_{H}5 domain could be obtained from 1 l E. coli culture using an oxidizing glutathione redox shuffle. V_{H}2, V_{H}4 and V_{H}6 could only be refolded using a redox shuffle with an excess of reduced glutathione and yielded about 0.2 mg soluble, refolded protein from 1 l E. coli. V_{H}1a, V_{H}1b, V_{H}3 and V_{H}5 remained in solution at 4° C. and no degradation was observed. In contrast, V_{H}2, V_{H}4 and V_{H}6 have a high tendency to aggregate upon standing at 4° C. Therefore, all subsequent experiments were performed with freshly purified proteins.

Analytical Gel Filtration

Samples of purified V_{H} domains were analyzed on a Superdex-75 column equilibrated with 50 mM Na-phosphate, pH 7.0, 100 mM NaCl, on a SMART-system (Pharmacia). The V_{H} domains were injected at a concentration of 2 μM in a volume of 50 μl, and the flow-rate was 50 μl/min. Lysozyme (14 kDa), carbonic anhydrase (29 kDa) and bovine serum albumin (66 kDa) were used as molecular standards.

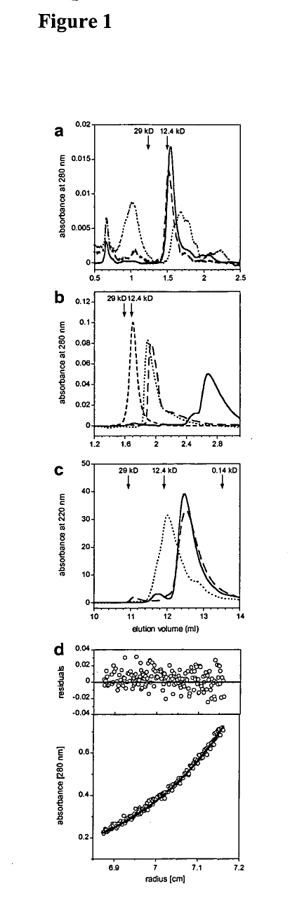

To analyze the oligomeric state of the purified domains in solution, analytical gel filtration experiments were performed. V_{H}1b, V_{H}3, and V_{H}5 elute at the expected size of a monomer (FIG. 1a with V_{H}3 as an example for monomeric V_{H} domains). V_{H}1a elutes under native conditions in three peaks that could not be assigned. We therefore investigated whether small amounts of denaturant might break up the aggregates. Using an elution buffer containing 0.5 M GdnHCl the unassigned peaks decrease and a peak at the size of a monomer showed up. With 0.9 M GdnHCl V_{H}1a elutes in a single peak corresponding to a monomer (FIG. 1b with the elution profile of a V_{H}1a at 0 and 0.9 M GdnHCl). V_{H}2, V_{H}4 and V_{H}6 did not elute from the column under native conditions. Even addition of 1.7 M GdnHCl to the elution buffer did not prevent these domains from sticking to the column. Elution could only be achieved with 1 M NaOH.

Equilibrium Denaturation Experiments of V_{H} Fragments

Fluorescence spectra were recorded at 25° C. with a PTI Alpha Scan spectrofluorimeter (Photon Technologies, Inc., Ontario, Canada). Slit widths of 2 and 5 nm were used for excitation and emission, respectively. Protein/GdnHCl-mixtures (2 ml) containing a final protein concentration of 0.5 μM and denaturant concentrations ranging from 0 to 5 M GdnHCl were prepared from freshly purified protein and a GdnHCl stock solution (7.2 M, in 50 mM NaPO₄, pH 7.0, 100 mM NaCl). Each final concentration of GdnHCl was determined from its refractive index. After overnight incubation at 10° C., the fluorescence emission spectra of the samples were recorded from 320 to 370 nm with an excitation wavelength of 280 nm. With increasing denaturant concentrations, the maxima of the recorded emission spectra shifted from about 342 to 348 nm. The fluorescence emission maximum was determined by fitting the fluorescence emission spectrum to a Gaussian function (isolated V_{H} domain and scFv fragments), or the fluorescence intensity at 345 nm (isolated V_{L} domains) was plotted versus the GdnHCl concentration. Protein stabilities for the isolated human consensus V_{H} and V_{L} domains were calculated as described (Jäger et al., 2001). To compare V_{H}, V_{L} and scFv denaturation curves in one plot, relative emission maxima and fluorescence intensities were scaled by setting the highest value to 1 and the lowest to 0.

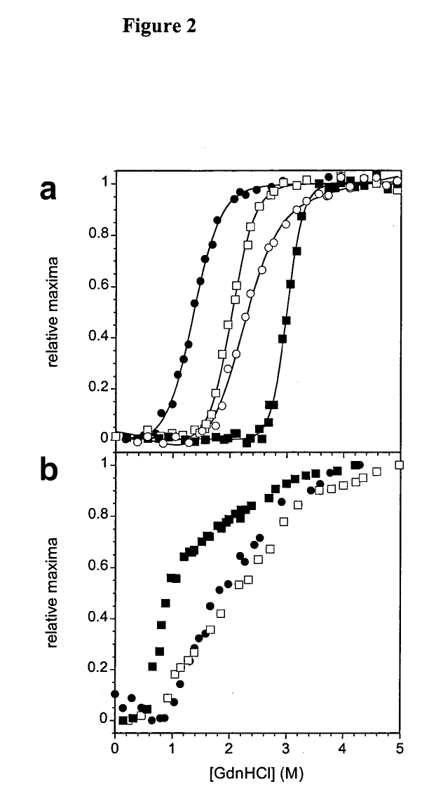

The thermodynamic stability of the seven human consensus V_{H} domains was examined by GdnHCl equilibrium denaturation experiments. Unfolding of the V_{H} domains was monitored by the shift of the fluorescence emission maximum as a function of denaturant concentration. FIG. 2a shows an overlay of the equilibrium denaturation curves of V_{H}1a, V_{H}1b, V_{H}3 and V_{H}5. In FIG. 2b the overlay is normalized to show the fraction of unfolded protein. The equilibrium denaturation of these domains is cooperative and reversible, which indicates two-state behavior. The V_{H}1a domain starts to unfold at 0.9 M GdnHCl, where V_{H}1a is monomeric in solution as indicated by gel filtration analysis. Therefore, the transition is only influenced by the stability of the monomeric V_{H}1a domain and not affected by multimerization equilibria. For the determination of free energy of unfolding the pretransition region of V_{H}1a, whose actual slope is influenced by the spectral changes caused by dissociation, was assumed to have the same slope and intercept as the V_{H}1b domain. V_{H}3 displays the highest change in free energy upon unfolding (ΔG_{N-U}) with 52.7 kJ mol⁻¹ and an unfolding cooperativity (m_{U}) of 17.6 kJ mol⁻¹ M⁻¹. V_{H}1b is of intermediate stability with a ΔG_{N-U} of 26.0 kJ mol⁻¹ and m_{U} of 12.7 kJ mol⁻¹ M⁻¹. V_{H}1a and V_{H}5 are less stable and have ΔG_{N-U} values of 13.7 and 19.1 kJ mol⁻¹ and m_{U} values of 10.1 and 8.6 kJ mol⁻¹ M⁻¹, respectively (Table 1). The range of m_{U} values can be compared to that expected for proteins of this size (14-15 kDa) and indicate that at least V_{H}1a, V_{H}1b, and V_{H}3 have the cooperativity expected for a two-state transition (Myers et al., 1995). The transition curves of V_{H}2, V_{H}4 and V_{H}6 in FIG. 2c show poor cooperativity, which indicates that no two-state behavior during GdnHCl equilibrium denaturation is followed. As the monomeric state of these V_{H} domains could not be ascertained, it is likely that part of this complicated transition involves the dissociation of multimers. The broad transition of V_{H}2 and V_{H}4 occurred between 1.0 and 2.5 M GdnHCl with a midpoint of 1.6 and 1.8 M GdnHCl, respectively. V_{H}6 shows a transition between 0.5 and 1.4 M GdnHCl with a midpoint of 0.8 M. This is the lowest midpoint of the examined domains, which indicates that V_{H}6 is the least stable human V_{H} domain.

Expression and Protein Purification of V_{L} Fragments

The four human consensus Vκ domains (Vκ 1, Vκ 2, Vκ 3 and Vκ 4) carrying the κ-like L-CDR3 from the antibody hu4D5-8 (sequence: HYTTP (Carter et al., 1992) were expressed in soluble form in the periplasm of E. coli. After purification with IMAC followed by a cation exchange column the Vκ domains could be obtained in high amounts, ranging from 17.1 mg/L bacteria culture normalized to an OD₅₅₀ of 10 for Vκ3 to 4.5 for Vκ1 (Table 1).

The κ-like L-CDR3 has a conserved cis-proline at position 136 (numbering scheme for variable domain residues according to Honegger & Plückthun, 2001). The amino acid sequence of Vλ domains never show a proline at this position. Therefore, we used for these domains a human consensus λ-like CDR3 (sequence: YDSSLSGV). The three human consensus Vλ domains (Vλ1, Vλ2 and Vλ3) were also expressed in soluble form in the periplasm of E. coli, but the yield after purification with IMAC and anion exchange column was much smaller than for the Vλ domains ranging from 1.9 mg/L bacteria culture normalized to an OD550 of 10 for Vλ2 to 0.3 mg for Vλ1 (Table 1).

Analytical Gel Filtration of V_{L} Fragments

While the monomeric V_{H} fragments elute at the expected molecular weight around 13 kDa (FIG. 1a), V_{L} domains in 50 mM sodium phosphate (pH 7.0) and 500 mM NaCl interact with different column materials. In the case of Vκ domains the best results could be obtained with a Superose-12 column (FIG. 1b). At a protein concentration of 50 μM, Vκ3 and Vκ2 elute at a molecular weight of 2 kDa, κ4 at 12 kDa and Vκ1 elutes with a broad peak even at the total volume of the column. Changing the concentration of Vκ4 from 50 to 5 μM, the peak shifts to a molecular weight of 2 kDa indicating a concentration dependent dimmer-monomer equilibrium under the assumption that Vκ domains eluting at 2 kDa are monomeric and at 12 kDa are dimeric (see below). Addition of 1 M GdnHCl or suggesting the NaCl concentration to 2M did not alter the elution profile. Vλ domains at concentrations of 5 μM show weakest unspecific interaction with silica based TSK columns (FIG. 1c) and Vλ1 and Vλ2 elute at a molecular weight of 7 kDa and Vλ3 elutes at an apparent molecular weight of 12 kDa.

To interpret these results from analytical gel filtration, the samples were also analyzed by equilibrium ultracentrifugation. The method was used to calibrate the elution values of the different columns for V_{L} domains: Vκ3 and Vλ2 give results consistent with a monomer, while λ3 shows a dimer (shown in FIG. 1d with Vκ3 as an example). Therefore, the V_{L} domains: Vκ2, Vκ3 and Vλ1 and Vλ2 eluting at an apparent molecular mass at 6 and 2 kD respectively, are indeed monomeric and the V_{L} domains: Vκ4 and Vλ3 eluting at 12 kDa are dimeric. Vκ1, which elutes even at the total volume of the column indicating a strong interaction with the column material, behaves in the ultracentrifugation as a monomer (Table 1).

Equilibrium Transition Experiments of V_{L} Fragments

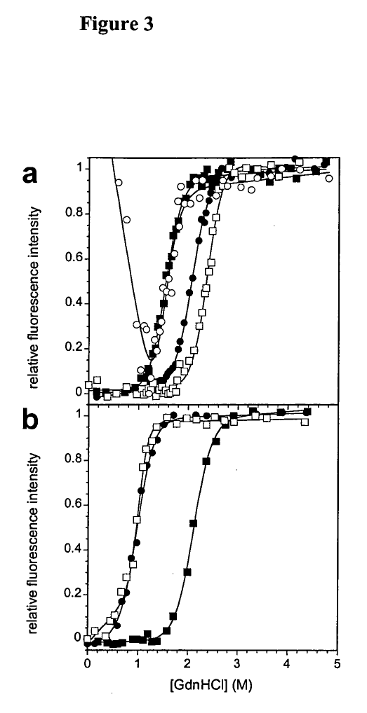

Most V_{L} domains have only one tryptophan (the highly conserved Trp43), which is buried in the core in the native state. In GdnHCl denaturation under native conditions no emission maxima could be determined, because the fluorescence is fully quenched by the disulfide bond Cys23-Cys106. During unfolding the tryptophan becomes solvent exposed, giving a steep increase in fluorescence intensity. Therefore, the thermodynamic parameters were calculated using the 6-parameter fit (Pace & Scholtz, 1997) on the plot of concentration of GdnHCl vs. fluorescence intensity, giving curves consistent with two-state behavior. All V_{L} domains show reversible unfolding behavior (data not shown). FIGS. 3(a) and 3(b) show relative fluorescence intensity plots against GdnHCl concentration of V_{κ} and V_{λ} domains. V_{κ}3 is the most stable V_{L} domain with a ΔG_{N-U} of 34.5 kJ mol⁻¹, followed by V_{κ}1 with 29.0 kJ mol⁻¹ and V_{κ}2 and V_{λ}1 with 24.8 and 23.7 kJ mol⁻¹, respectively (Table 1). The least stable V_{L} domains are V_{λ}2 and V_{λ}3 with a ΔG_{N-U} of 16.0 and 15.1 kJ mol⁻¹. All V_{L} domains show m-values between 11.1 and 16.2 kJ mol⁻¹ M⁻¹, indicating that they have the cooperativity expected for a two-state transition (Myers et al., 1995). The human consensus V_{κ}4 carries an exposed tryptophan at position 58 in addition to the conserved Trp43, which is not quenched in the native state. The denaturation curve is fully reversible, but shows a steep pre-transition baseline followed by a non-cooperative transition. Because of this uncertainly, no ΔG_{N-U} values for V_{κ}4 but only the midpoint of transition are reported, which is at 1.5 M GdnHCl. For the V_{κ}4 domain Len, a stability of 32 kJ/mol has been reported (Raffen et al., 1999).

Analysis of Primary Sequence and Model Structures

In the group of isolated V_{H} fragments large differences are seen: V_{H}3 shows the highest yield of soluble protein and thermodynamic stability, V_{H}1a, V_{H}1b and V_{H}5 show intermediate yield and intermediate or low stability, while V_{H}2, V_{H}4 and V_{H}6 show more aggregation prone behavior and low cooperativity during denaturant-induced unfolding. The properties of V_{κ} and V_{λ} domains are more homogenous. The thermodynamic stabilities differ by only approximately 10 kJ/mol in the group of V_{κ} and in the group V_{λ} domains. In general, the stability and soluble yield is higher in isolated V_{κ} domains than in V_{λ} domains. To analyze possible structural reasons for this different behavior of the variable antibody domains, the primary sequence and the modeled structures of the seven human consensus V_{H} and V_{L} domains were analyzed. The models have been published previously (Knappik et al., 2000) (PDB entries: 1DHA (H1a), 1DHO (H1b), 1DHQ (H2), 1DHU (H3), 1DHV (H4), 1DHW (H5), and 1DHZ (H6)) and V_{L} domains (PDB entries: 1DGX (κ1), 1DH4 (κ2), 1DH5 (κ3), 1DH6 (κ4), 1DH7 (λ1), 1DH8 (λ2), 1DH9 (λ3)). The quality of the models varies for the different domains. Many antibody structures in the Protein Data Bank use, for example, the V_{H}3 framework, and the chosen template structure for building the model shares 86% sequence identity excluding the CDR3 region (PDB entry: 1IGM) and the structural differences between templates could be traced to distinct sequence differences. In the case of V_{H}6, the closest templates were human V_{H}4 and murine V_{H}8 domains, since no crystal structure of a member of the V_{H}6 germline family is available in the PDB. Both germline families encode a different framework 1 structural subtype (I) than V_{H}6 (III) (Honegger & Plückthun, 2001). The chosen template for V_{H}6 (PDB entry: 7FAB) shares 62% sequence identity, excluding the CDR3 region and belongs to human V_{H}4. Three questions regarding the domains in isolation came up: Why is V_{H}3 so extraordinarily stable, why do V_{H}2, V_{H}4 and V_{H}6 behave comparatively poorly concerning expression and aggregation and why did V_{κ} domains give higher yields and are more stable than V_{λ} domains?

Salt Bridges

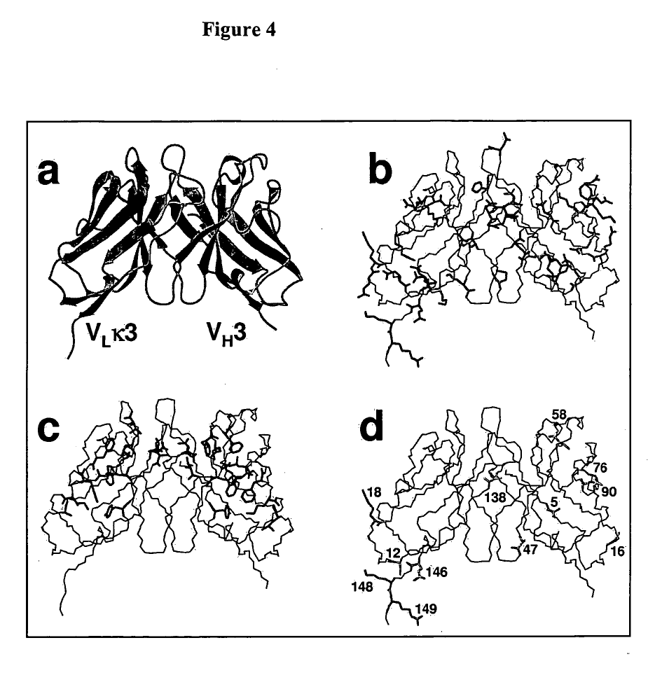

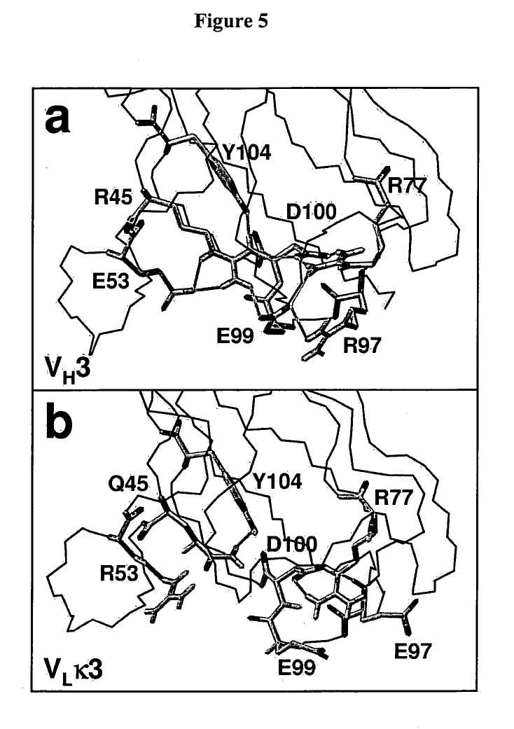

Salt bridges between positively and negatively charged amino acids and repulsions between equally charged amino acids play an important role in protein stability (Nakamura, 1996). FIG. 4a shows a schematic representation of a scFv fragment consisting of V_{L}κ3 and V_{H}3 domain with its characteristic secondary structure. In FIG. 4b positively charged residues of at pH 7.0 are shown in gray and negatively charged residues are shown in black. There is an accumulation of charged residues at the base of the domain. In V_{H} domains, the conserved residues Arg45, Glu53, Arg77, and Asp100 form buried conserved salt bridges connecting Arg45-Glu53, Arg45-Asp100, and Arg77-Asp100 (FIG. 5a). At position 77 the V_{H}5 consensus is Gln instead of Arg of the consensus of the other subfamilies (Table 2). This change results in loss of the conserved salt bridge connecting Arg77 and Asp100. In addition, charged residues at positions 97 and 99 can be part of the charge cluster. Only V_{H}1a, V_{H}1b, V_{H}3, and V_{H}6 have Glu at position 99. These domains can form additional salt bridges between Glu99-Arg45, as seen in the structure with PDB entry 1IGM or between Glu99-Arg77 as seen in structures with PDB entries 1BJ1, 1INE, 2FB4 and 1VGE.

In V_{L} domains (FIG. 5(b)) the amino acid at position 45 is uncharged and the ones in position 53 and 97 are either reversed compared to the amino acids at these positions in V_{H} domains or are uncharged. Therefore, the charge cluster contains only one conserved salt bridge connecting Arg77 and Asp100 and one main-chain side-chain hydrogen bond connecting Glu97 and Arg77 (FIG. 5(b)). The least stable V_{κ} domain V_{κ}2 carries Leu at position 45, which is unable to form a side-chain side-chain hydrogen bond to Tyr104, which is conserved in the other V_{L} domains and also in V_{H} domains (FIGS. 5(a) and (b)).

Hydrophobic Core Packing

Another important stabilizing factor is hydrophobic core packing (Pace, 1990). All model structures were checked for cavities, which would indicate improper packing leading to fewer van der Waals interactions and reduced thermodynamic stability. A van der Waals contact surface was generated for a water radius of 1.4 Å with the program Molmol (Koradi et al., 1996). When cavities were found, the surrounding residues were checked whether they would contribute hydrophobic surface area to the cavity. A cavity lined with hydrophobic residues would be less favorable as a water molecule would be energetically unfavorable at such a position. Based on these cavities and sequence comparisons between the different variable domain frameworks, positions in the hydrophobic core could be identified, which may lead to sub-optimal packing. In FIG. 4C, an overview of the analyzed core residues is given. The core residues are divided into two regions: the upper and lower core according to the orientation shown in FIG. 4a. The upper core is build of buried residues above Trp43, the conserved disulfide bridge between Cys23, and Cys106 and Gln/Glu6 towards the CDRs. Part of the CDR residues are involved in the upper core with the consequence that different CDRs have a strong influence on the upper core (and its contribution to the overall stability) and vice versa the residues of the upper core an influence on the conformation of the CDRs (and affinity or specificity of antigen binding) (Eigenbrot et al., 1993). The lower core is below Trp43 and its conformation is related to the type of amino acid at position 6, 7, 10 and 78 (Saul & Poljak, 1993).

Upper Core

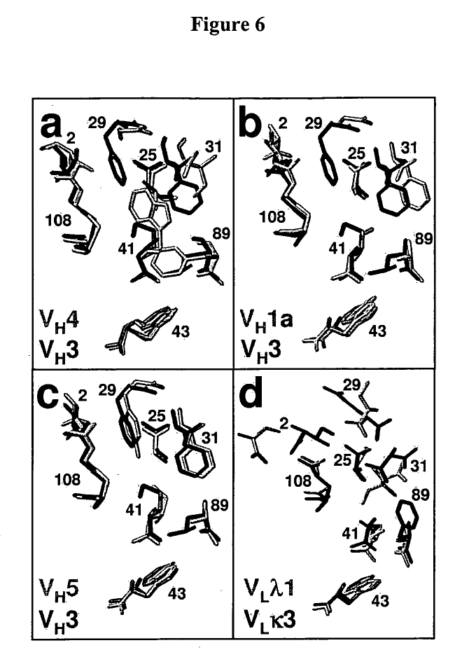

The residues 2, 4, 25, 29, 31, 41, 80, 82, 89, and 108 form the upper core. In the sequence alignment shown in Table 2 these residues have been compared for the variable domains. In V_{H} domains two sequence motifs can be distinguished: the V_{H}3-like motif with two bulky aromatic residues at positions 29 and 31 (V_{H}1b, V_{H}3, V_{H}5), the alternative location of the aromatic residues at 25 and 29 (V_{H}2) and the V_{H}4/V_{H}6 motif with Trp at position 41 and a big aliphatic residue at position 25. FIG. 6(a) shows a superposition of V_{H}4 on V_{H}3, highlighting the differences between these motifs. In the V_{H}3-like motif Phe29 and Phe31 fill the space between the neighboring residues 2, 25, 31 and 108. In the V_{H}4/V_{H}6 motif, these two residues are changed to smaller residues. Here Trp41 and the methyl group of Val25 fill up the empty space. V_{H}1a belongs to the V_{H}3-like motif but has a Gly instead of Phe at position 29. No other residue compensates for this empty space, which results in a hydrophobic cavity (FIG. 6(b)). V_{H}1a, V_{H}1b and V_{H}5 have an Ala instead of a Leu (V_{H}3) at position 89. There is no obvious compensation for this loss of an isopropyl group. In addition, the substitution of Ala25 (V_{H}3) to Gly in V_{H}5 (Table 2) equals the loss of a methyl group, further weakening the packing of the upper core of V_{H}5 (FIG. 6(c)).

FIG. 6(d) shows the superposition of the upper core of the V_{κ}3 and V_{λ}1 domain as representatives of V_{κ} and V_{λ} domains. The packing density of the V_{κ} domains compared to the V_{H} domains is smaller, because there is only one bulky aromatic amino acid in the upper core of V_{κ} domains at position 89, compared to V_{H} domains that have at least two aromatic residues (Table 2). The packing density is further lowered in V_{λ} domains because of the smaller Gly in position 25 and Ala in position 89 instead of Ala/Ser and Phe, respectively, which are found in V_{κ} domains (FIG. 6(d), Table 2), consistent with a lower thermodynamic stability of V_{λ} domains.

Lower Core

Within V_{H} domains an interesting correlation is seen between stability and framework 1 classification after Honegger and Plückthun (Honegger & Plückthun, 2001), which influences hydrophobic core packing of the lower core (Saul & Poljak, 1993) and is determined by the type of amino acid in positions 6, 7 and 10 (Table 3). The most stable V_{H}3 domain falls into subgroup II, while V_{H}1a, V_{H}1b and V_{H}5 with intermediate properties fall into subgroup III (Table 3). The V_{H} domains showing high inclusion body propensity and no cooperative denaturation V_{H}2, and V_{H}4 fall into subgroup I. V_{H}6 is a member of subgroup III because of its Gln at position 6 and the absence of Pro in position 7. However, previous experiments (Jung et al., 2001) have shown that Pro in position 10 destabilizes the domain.

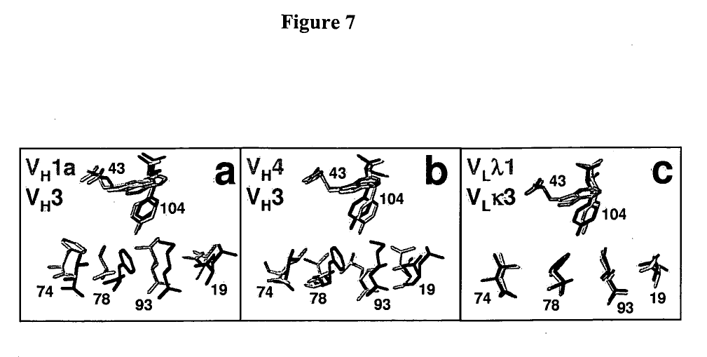

Residues 19, 74, 78, 93, and 104 (Table 2) are part of the lower core, which is built of residues 13, 19, 21, 45, 55, 74, 77, 78, 91, 93, 96, 100, 102, 104 and 145. Only V_{H}3, the most stable framework, has a bulky aromatic residue (Phe) at position 78. However, V_{H}1a, V_{H}1b, and V_{H}5 have Phe at position 74, thereby simply switching the residues in positions 74 and 78, probably leading to similar interactions (FIG. 7(a)). VH5 has an additional exchange at position 93 from Met to Trp. This additional aromatic residue in V_{H}5 could help compensate for the loss of Phe78 and the poor interactions in the charge cluster (see above). Apart from Tyr104, no additional aromatic residue stabilizes the lower core of V_{H}2, V_{H}4, and V_{H}6 (FIG. 7(b)).

In V_{L} domains only one framework 1 subtype is found (Honegger & Plückthun, 2001), and as a consequence, the lower core residues of V_{κ} and V_{λ} domains are almost the same and have similar orientations (Table 2 and FIG. 7).

Residues Possibly Influencing Solubility and Folding Efficiency

Residues that could correlate with poor expression behavior and a high tendency to aggregate due to kinetic rather than thermodynamic reasons (Fink, 1998) were further examined. The analysis was started from a sequence alignment of the human consensus V_{H} domains grouped by V_{H} with good biophysical properties (V_{H}1a, V_{H}1b, V_{H}3, V_{H}5) and more aggregation prone V_{H} domains (V_{H}2, V_{H}4, V_{H}6) (Table 3).

It was shown previously that mutations of exposed hydrophobic residues do not change the solubility of the native scFv fragment, as determined by salting-out, but have a profound effect on the in vivo folding yield (Nieba et al., 1997). Position 5 is exposed to solvent and therefore the hydrophilic residue Gln or Lys of V_{H}2, V_{H}4, and V_{H}6 might be thought to decrease the aggregation tendency in contrast to the hydrophobic Val in V_{H}1a, V_{H}1b, V_{H}3, and V_{H}5. Nevertheless, in a selection experiment favoring stability (Jung et al., 1999), Val was selected out of Val, Gln, Leu, and Glu in the scFv 4D5Flu, possibly indicating the importance of local secondary structure propensity.

V_{H}2, V_{H}4 and V_{H}6 have a non-glycine residue with a conserved positive phi angle at position 16 (FIG. 4(d)), which causes an unfavorable local conformation. Structures that have been determined with a non-Gly residue at position 16 (e.g. PDB entries 1C08, 1DQJ, 1F58) indeed show that the positive phi angle is locally maintained, apparently enforced by the surroundings. In contrast, the odd-numbered V_{H} have all Gly at this position.

For the antibody McPC603, it has been shown by Knappik & Plückthun, 1995 that the exchange of Pro47 to Ala, adjacent to another Pro at position 48, does not result in better thermodynamic stability, but enhances folding efficiency. V_{H}2 and V_{H}4 also carry Pro at position 47. In V_{H}6, the highly conserved hydrophobic core residue Ile is exchanged to Thr at position 58, which buries an unsatisfied hydrogen bond donor.

A proline residue in position H10 can have a strong influence on FR 1 conformation. V_{H} structures can be classified into four subtypes with distinct FR 1 conformation and correlated differences in the packing of the lower core depending on the type of amino acid found in positions H6, H7 and H10 (Honegger & Plückthun, 2001a). To prove that these residues indeed cause the different conformations, Jung et al. (2001) introduced different H6/H7/H10 residue combination into the same V_{H} domain and determined the effect on the structure by X-ray crystallography. In their system, all combinations containing Pro in position 10 were destabilized compared to molecules containing a Gly, Ala or Ser in this position. While these constructs contained Pro in an “unnatural” combination with a V_{H}-domain normally containing a different amino acid in this position, and therefore the destabilizing effect could also be due to a mismatch between local sequence and overall sequence context, the poorly behaved V_{H}2, V_{H}4 and V_{H}6 all contain Pro10, while V_{H}1B, V_{H}1B, V_{H}3 and V_{H}5 have a Gly or Ala in this position.

At position 44 the even numbered V_{H} domains carry Ile in contrast to Val of the odd numbered V_{H} domain. This position is located at the interface to V_{L} and should have no effect on the isolated domains, but it should have an effect when in complex with V_{L}.

The exposed CDR 2 residue 60 of the even numbered V_{H} domains is an aromatic bulky amino acid (Trp and Tyr) and probably decreases folding efficiency. This residue cannot be exchanged because of possible participation in antigen binding.

The solvent exposed residue 72 was changed in the antibody McPC603 from a hydrophobic residue Ala to Asp, which increases the soluble/insoluble ratio 20-fold but does not alter the thermodynamic stability (Knappik et al., 1995). V_{H}6 carries a hydrophobic Val at this position.

The odd numbered V_{H} domains have Gly at position 76 in contrast to the even numbered V_{H} domains, which carry Thr or Ser. In half of the antibody structures determined that are found in the PDB the residue at this position has a positive phi angle, indicating that glycine could be better at this position.

The semi-buried position 90 of V_{H}1a, V_{H}1b, V_{H}3, and V_{H}5 is occupied with Tyr, whereas V_{H}2, V_{H}4, and V_{H}6 have Val or Ser. The influence of this substitution on the poor behavior of the even numbered domains can only be tested experimentally.

As the V_{L} domains can be primarily grouped in κ and λ domains the analysis was concentrated on a comparison between these two groups. At the solvent exposed C-terminal end at positions 146, 148 and 149 V_{κ} domains have charged amino acids in contrast to V_{λ} domains, which have Thr, Leu and Gly, respectively, at these positions (Table 4, FIG. 4(d)). In addition, the hydrophilic Thr in position 138 of κ domains is exchanged to the hydrophobic Val in λ domains (Table 4, FIG. 4(d)). These exchanges of less hydrophilic residues in V_{λ} domains possibly lower the folding efficiency of these domains and may be a contributing factor to the smaller soluble yield compared to V_{κ} domains.

Proline is an α-helix and β-strand breaker and thus destabilizes those secondary structures. Positions 12 and 18 in V_{L} domains are both part of a β-sheet structure. Only V_{κ}2 has Pro at both positions while Ser and Arg, respectively, are the dominant residues at these positions in the other V_{L} domains (Table 4, FIG. 4(d)).

Expression and Protein Purification of scFv Fragments

After biophysical characterization of isolated human consensus V_{H} and V_{L} domains systematic combinations of V_{H} and V_{L} were also tested to understand their mutual influence on biophysical properties and chose the scFv format, in which the V_{H} domain is linked via a flexible peptide linker to the V_{L} domain. To limit the number of possible V_{H}-V_{L} combinations of 49, the scFv fragments with the most stable V_{H} domain V_{H}3 was tested combined with each of the seven human consensus V_{L} domains and, conversely, the most stable V_{L} domain V_{κ}3 with each of the seven human consensus V_{H} domains. It should be examined if there is a mutual compensation or addition of the individual biophysical properties of the isolated variable domains in the scFv fragment or if even synergetic effects can occur.

All V_{H} domains within the scFv fragment carry the same H-CDR3, which is derived from the V_{H} domain of the well expressing antibody 4D5 (Knappik et al., 2000; Carter et al., 1992). The V_{κ} and V_{λ} domains in the scFv fragments carry the κ- and λ-like L-CDR3, respectively. All scFv fragments could be expressed in soluble form in the periplasm and purified with IMAC, followed by an anion exchange column. Purity of the fragments was over 98%, confirmed by SDS-PAGE analysis (data not shown) and the subsequent measurements were all carried out with freshly purified proteins. To compare the expression yield of the scFv fragments with the different V_{H} or V_{L} domains, we additionally isolated the scFvs with a batch method. To test the error inherent in the yield determination the scFv H3κ3 was purified 4 times independently. The yield of purified H3κ3 was 6.5±0.2 mg from a 1 L bacteria culture normalized to an OD₅₅₀ of 10, which is approximately the final cell density in a shaken flask under these conditions. Yields of all scFv fragments tested were normalized to the yield of H3κ3 and were in the range of 2.6 to 12.4 mg/L (Table 5). H1aκ3 and H1bκ3 with 11.1 mg/L and 12.4 mg/L, respectively, (1.7 and 1.9 fold the amount of H3κ3), show the highest yield and H2κ3, H4κ3 and H6κ3 show the lowest yield of scFv fragments with the V_{κ}3 domain with 0.6, 0.4 and 0.6 fold that of H3κ3, respectively. All scFv fragments with V_{H}3 but different V_{L} domains show yields only below that of H3κ3. The percentage of insoluble protein was determined for H3κ3 in 4 independent measurements to be (30±10) %. The other scFv fragments tested show a percentage of insoluble protein between 50% and 10% with the exception of H2κ3, H4κ3 and H6κ3, which show a percentage of insoluble protein between 80% and 90% (Table 5).

Analytical Gel Filtration of scFv Fragments

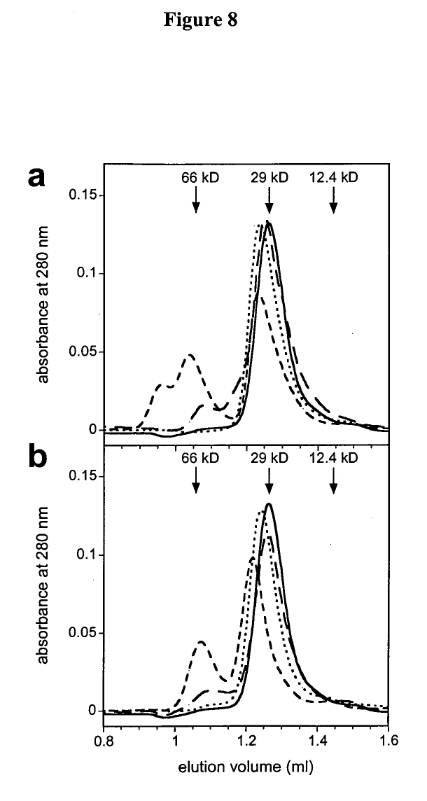

H3κ3 elutes from an analytical gel filtration column Superdex-75 at a protein concentration of 5 μM in 50 mM sodium phosphate (pH 7.0) and 500 mM NaCl with an apparent molecular weight of 29 kDa, which indicates that H3κ3 is monomeric in solution. The other scFv fragments with V_{L}κ3 as the V_{L} domain are also monomeric under these conditions, with the exception of H1aκ3, which shows besides the monomer peak also smaller dimer and multimer peaks. H4κ3 shows in addition a small amount of dimer of less than 10%. FIG. 8(a) shows the chromatogram of H3κ3 as an example for monomeric scFv fragments, along with H1aκ3 and H4κ3. The scFv fragments with V_{H}3 and a V_{κ} domain are all monomeric whereas H3κ1 shows in addition a small dimer peak (FIG. 8(b) with H3κ3 as an example for monomeric scFv fragments and H3κ1). In contrast, the scFv fragments with V_{λ} domains all show monomer-dimer equilibria, with a dimer content from 20% in the case of H3λ1 to 70% in the case of H3λ2 (FIG. 8(b) with H3λ1 as an example for scFv fragments with a V_{λ} domain). With 1 M GdnHCl in the elution buffer all those scFv fragments, which had a dimer fraction under native conditions, elute in a single peak at an apparent mass of 29 kDa, indicating that they are now fully monomeric. The chromatogram in 1 M GdnHCl is shown in FIG. 8(a) for H1aκ3 and in FIG. 8(b) for H3λ1 as an example for scFv fragments with V_{λ} domain. It should be noted that this concentration is below the major transition of all scFv fragments. The only exception was H3λ2, which still has dimer content of 20% in 1 m GdnHCl. With 2 M GdnHCl, also H3λ2 shows only a monomer peak (data not shown).

Equilibrium Unfolding Experiments of scFv Fragments

Unfolding and refolding of the scFv fragments as a function of denaturant concentration was monitored by the shift of the maximum of the fluorescence emission after excitation at 280 nm. Each scFv fragment shows reversible unfolding behavior (data not shown). The denaturation of the scFv fragments is usually not a two-state process (Wörn & Plückthun, 2001), because the scFv fragments are built from two domains, which may have different intrinsic stabilities and interact over an interface region and can potentially stabilize each other. Therefore, no ΔG_{N-U} values are reported, but instead the midpoints of the transitions of denaturation are given, which are a semi-quantitative measure for the stability of the scFv fragments. The assignment of the transitions to V_{H} or V_{L} domain results from the determination of the transition of single domains (Table 1). In Table 5 the midpoints are listed for the V_{H} and V_{L} domain within the scFv fragments. If only one transition is visible, the midpoint is assigned to both the V_{H} and V_{L} domain.

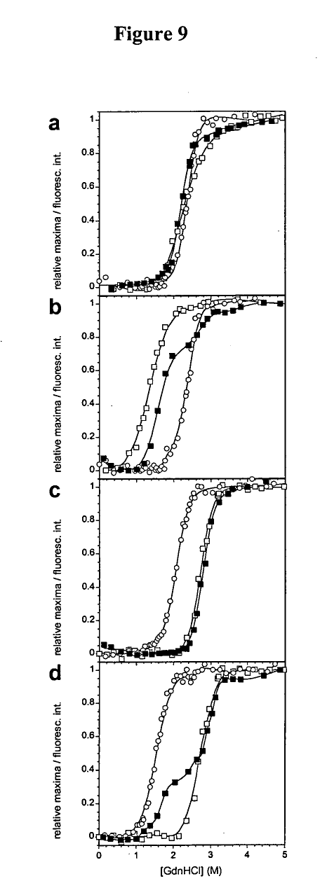

With the knowledge of the denaturation properties of the isolated V_{H} and V_{L} domains and the combinations of these domains in the scFv fragments it is now possible to systematically study the influence of the interface interaction on the stability of the scFv fragments. Different cases can be distinguished (Wörn & Plückthun, 1999): If the stability of the isolated V_{H} and V_{L} domains is very similar, the resulting scFv has also the same stability (see FIG. 9(a) with H5κ3 as an example). If one domain is significantly more stable than the other, the less stable one can be stabilized through the interface interaction with the other domain (see FIG. 9(b) with H1aκ3 with the more stable V_{κ}3 stabilizing V_{H}1a, and FIG. 9(c) with H3κ1 with the more stable V_{H}3 stabilizing V_{κ}1). Nevertheless, it is also possible that, although the stability of the domains is different, almost no stabilization of the less stable domain occurs (see FIG. 9(d) with H3κ2 as an example).

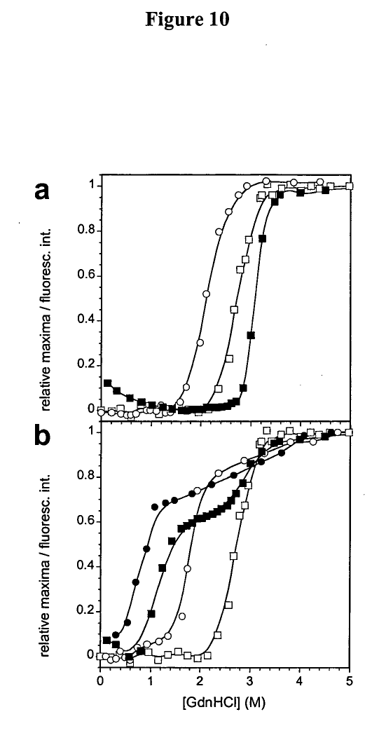

The scFv fragments with V_{λ} domains show an interesting behavior (FIG. 10(a) with H3λ1 as an example) because the scFv fragments are even more stable than any of the single isolated domains. Apparently, the interface interaction between V_{H} and V_{L} is so strong that the domains are stabilized above the intrinsic stability of the isolated domains. If the interface finally breaks up, the now isolated domains in the scFv unfold directly, explaining the steep transition. This extraordinary behavior strongly depends on the sequence of L-CDR3.

V_{λ} domains were also cloned and purified with the κ-like L-CDR3. The isolated V_{λ} domains with the κ-like CDR3 gave very poor yields. They do not show reversible behavior in denaturant induced equilibrium denaturation and have lower midpoints of denaturation than the corresponding V_{λ} domain with the λ-like L-CDR3. The combinations of V_{H}3 with V_{λ} domains carrying the κ-like CDR3 show similar yield and dimer/monomer ratios in analytical gel filtration as the ones carrying the λ-like CDR3 (data not shown) but a different behavior in GdnHCl denaturation. As an example, FIG. 10(b) shows H3λ1 with a κ-like L-CDR3, where the V_{λ}1 domain is only slightly stabilized in comparison to the renaturation curve of the isolated V_{λ}1, indicating that the interface stabilization in this case is not so strong. It should be noted that the only difference between the two scFv fragments in FIGS. 10(a) and (b) is the different L-CDR3, which obviously causes this dramatic stabilization difference. The κ-like CDR3 with proline in position 136 builds a rigid Ω-loop, which probably interferes with the perfect orientation between V_{H} and V_{L}.

In summary, the most stable scFv fragments found to denature only starting above 2 M GdnHCl are H3κ3, H1bκ3, H5κ3 and H3κ1. Although the isolated V_{λ} domains are rather unstable by themselves, in combination with V_{H}3 they can build very stable scFv fragments, but depend on the L-CDR3 for this effect. Most likely this CDR is responsible for a favorable orientation of V_{L} to V_{H} and thus enables a tighter interaction through the interface. ScFv fragments with an intermediate stability starting denaturation above 1 M GdnHCl are H1aκ3, H2κ3, H3κ2 and H3κ4, while H4κ3 and H6κ3 are scFv fragments with a modest stability, starting denaturation under 1 M GdnHCl.

### Example 2

Structure-Based Improvement of the Biophysical Properties of Immunoglobulin V_{H} Domains with a Generalizable Approach

### Abbreviations

CDR, complementary determining region; GdnHCl, guanidine hydrochloride; HuCAL, Human Combinatorial Antibody Library; IMAC, immobilized metal ion affinity chromatography; IPTG, isopropyl-β-D-thiogalactopyranoside; scFv, single-chain antibody fragment consisting of the variable domains of the heavy and of the light chain connected by a peptide linker; V_{H}, variable domain of the heavy chain of an antibody; V_{L} variable domain of the light chain of an antibody.

In a systematic study of V gene families carried out with consensus V_{H} and V_{L} domains alone and in combinations in scFv fragments, we found comparatively low expression yields and lower cooperativity in equilibrium unfolding in antibody fragments containing V_{H} domains of human germline families 2, 4 and 6. From an analysis of the packing of the hydrophobic core, the completeness of charge clusters, the occurrence of unsatisfied hydrogen bonds, and residues with low β-sheet propensity, positive Φ angle and exposed hydrophobic side chains, we pinpointed residues potentially responsible for these unsatisfactory properties of these germline-encoded sequences. Several of those are in common between the domains of the even-numbered subgroups, but do not occur in the odd-numbered ones. In this study, we have systematically exchanged those residues alone and in combination in two different scFv fragments using the V_{H}6 framework and we describe their effect on equilibrium stability and folding yield. We improved the stability by 20.9 kJ/mol, the expression yield by a factor 4, and can now use these data to rationally engineer antibodies derived from this and similar germline families for better biophysical properties. Furthermore, we provide an improved design for libraries exploiting the significant additional diversity provided by these frameworks. Both antibodies studied here completely retain their binding affinity, demonstrating that the CDR conformations were not affected.

Recombinant antibodies are used in an ever increasing number of applications from biological research to therapy. In addition to showing high antigen specificity and affinity, such recombinant antibodies should also be obtainable in high yield, have low tendency to aggregate and be stable against high denaturant concentrations, elevated temperatures and proteases, depending on the requested task. A popular format for many of these applications is the single-chain Fv (scFv) fragment, where the variable domain of the heavy chain (V_{H}) is connected via a flexible linker to the variable domain of the light chain (V_{L}) or vice versa (1-3). This format contains the complete antigen binding site and can be expressed in a wide range of hosts including bacteria (4) and yeast (5). While we chose to investigate these questions with scFv fragments, as their simple structure makes an untangling of domain interactions much easier, differences in physical properties are also manifest in Fab fragments and whole antibodies, which contain the same domains.

Mutations important for the biophysical behavior can either influence the equilibrium thermodynamic stability or the aggregation tendency during folding or both. While these properties are distinguishable and mutations are known (see below) which influence only one of these properties, frequently they are related and amino acid exchanges can have an effect on both. Mutations influencing thermodynamic stability can make contributions to many different types of interactions, such as packing of the hydrophobic core, secondary structure propensity, charge interactions, hydrogen bonding, desolvation upon unfolding, compatibility with the enforced local structure, and many more (6, 7). Mutations that influence folding efficiency can also be part of this list, as the stability of intermediates is an important component. Additionally, however, natural proteins use “negative design” (8) to avoid aggregation. In its simplest form, this avoids hydrophobic patches on the surface. In the case of antibodies, such hydrophobic patches were found to have almost no effect on the solubility of the native protein, correctly defined as the maximal concentration of the soluble native protein (9). The hydrophobic patches can have a very dramatic effect on the folding yield and thus the yield of functional protein in E. coli, which is colloquially but incorrectly often termed “solubility”, as the yield describes the overall process of producing soluble protein, but not its solubility.

In the case of scFv fragments, a further complication is introduced by their two-domain nature. The two domains can stabilize each other and unfold either cooperatively or with an equilibrium intermediate, depending on the relative intrinsic stability of the domains and their interface (10). However, from these studies of domain interactions and a systematic study of isolated domains and their interactions (see Example 1, 11), we can now untangle this system. We can thus pinpoint the problem spots, and in the present study we wish to provide the evidence that a correction of these small defects indeed leads to a marked improvement of phenotypes.

It is thus important to distinguish expression yield from thermodynamic stability. In the periplasmic expression of antibodies, the most important limitation of the level of observed expression level of functional protein is the periplasmic folding yield (4). Antibodies with poor yield of functional protein give rise to periplasmic aggregates. There are three principal mechanisms leading to an increased expression yield of soluble proteins: Increasing the total expression level (provided the folding yield stays constant), increasing the folding yield in E. coli or decreasing degradation by E. coli proteases. All three mechanisms can be somewhat influenced by extrinsic factors including the choice of bacterial strain, expression vector, media composition, and expression temperature (summarized in ref. (4)) and coexpression of periplasmic chaperones (12, 13). Nevertheless, the major contribution to changes of the expression yield of folded protein is due to changes in the protein sequence itself. In the case of secreted proteins placed in the same vector, the translation initiation region and the beginning of the protein sequence (the signal sequence) is identical between different variants. Therefore, sequence changes are extremely unlikely to influence translation per se. Mutations leading to higher thermodynamic stability often also decrease protease digestion of the protein, as the E. coli proteases usually prefer unfolded protein as a substrate. Nevertheless, mutations removing potential cutting sites for E. coli proteases may also prevent degradation. Mutations may thus also influence the efficiency of folding, independent of influencing the equilibrium thermodynamic stability of the protein. Side reactions of the folding process often lead to aggregated protein, which is enriched in inclusion bodies. The kinetic partioning into productive folding and aggregation can be influenced by mutations increasing either the thermodynamic stability of intermediates or removing a solvent-exposed hydrophobic residue or otherwise making the surface less suitable for aggregate growth (“negative design” (8)). In addition, the mutations increasing folding efficiency can also indirectly lead to a higher total expression level by preventing the formation of toxic side-products, most likely soluble aggregates, which lead to leakiness of the outer membrane and eventually decrease the viability of E. coli.

There are different approaches finding residues that improve the thermodynamic stability and yield of soluble protein of scFv fragments (reviewed by Wörn & Plückthun (7)). Previously, most work had concentrated on the optimization of individual antibodies. If the three-dimensional (3D) structure of the antibody to be improved is known, a detailed analysis can identify problematic residues, which can then be exchanged by side-directed mutagenesis (14-16). A second approach uses random mutagenesis followed by selection with a bias toward the improvement of the desired property (17-19). The consensus approach as a third approach (20) uses the sequence information from antibodies naturally encoded by the immune system. The genes of immunoglobulin variable domains, as is assumed for all gene families, have diverged by multiple gene duplications and mutations. Selected genes are further subjected to an accelerated “local” evolution by somatic mutations that optimize the capacity of the antibody to bind to antigen structures with high affinity, but these mutations are not propagated in the germline. In contrast, mutations acquired during the duplication of the primordial V gene to make the present-day Ig-locus are manifest as germline family-specific differences. In this study, we wanted to explore a generic approach for improving antibodies for their biophysical properties combining the above knowledge with our knowledge of the biophysical properties of the germline-encoded V_{H}, V_{κ} and V_{λ} families (see Example 1, 11). Since we focus on genes with initially germline-encoded sequences, our approach is not limited to improving individual molecules and thus to removing changes introduced by somatic mutations, but particularly to problematic residues encoded by different germline genes.

Destabilizing mutations may be highly probable but are selectively neutral as long as the overall domain stability does not fall below a certain threshold (20). Conversely, random mutations resulting in increased thermodynamic stability are highly improbable in the absence of a positive selection. Consequently, the most frequent amino acid at any position in an alignment of homologous immunoglobulin variable domains should be most favorable for the stability of the protein domain. This method was tested on a V_{κ} domain and of ten proposed mutations six increased the stability. Nevertheless, the simplification inherent in this approach is that all frameworks are averaged to a single “ideal” sequence. The different germline genes or frameworks have an important function for antibody diversity. First, framework residues in the outer loop and close to the 2-fold axis can contribute important interactions to protein- and hapten-antigens, respectively. Second, several framework regions can influence the conformation of the CDRs and thereby indirectly modulate antigen binding. Third, different frameworks carry mutually incompatible residues, which cannot simply be exchanged to those of other frameworks. It follows that family-specific solutions are needed to create a variety of different frameworks with superior properties. In this paper we provide the basis for this approach.

Recently, we analyzed the biophysical properties of human germline family-specific consensus domains (see Example 1, 11) derived from the Human Combinatorial Antibody Library (HuCAL™) (21). In case of the V_{H} domains we found that the V_{H}3 germline family-specific consensus domain was the most stable V_{H} domain, followed by the V_{H}1a, V_{H}1b and V_{H}5 consensus domains with intermediate stabilities and only little or no aggregation-prone behavior. V_{H}2, V_{H}4 and V_{H}6 domains, on the other hand, showed low cooperativity during denaturant-induced unfolding, lower yield and a higher tendency to aggregate. The detailed analysis of hydrophobic core packing and formation of salt bridges revealed that the V_{H}3 domain had always found the optimal solution while all other V_{H} domains had some shortcomings explaining the higher thermodynamic stability of V_{H}3. Furthermore, with the help of a sequence alignment grouped by V_{H} domains with favorable properties (families 1, 3 and 5) and unfavorable properties (families 2, 4 and 6), residues of the even-numbered V_{H} domains were identified and structurally analyzed which potentially decrease the folding efficiency being the reason for the unfavorable properties.

In this study, we used a structure-based approach exploiting the knowledge of the biophysical properties of the human germline family-specific consensus V_{H} domains (see Example 1, 11), and in addition, resorting to tables of published and in-house selection experiments (A. Honegger et al., unpublished) to improve the V_{H}6 framework as a model. We chose the V_{H}6 framework, because it shows a somewhat aggregation-prone behavior and the lowest midpoint of denaturation, compared to the other human V_{H} domains, indicating that V_{H}6 is the V_{H} domain with the lowest thermodynamic stability. These properties were observed with isolated domains as well as in the scFv format with V_{κ}3 (see Example 1, 11). We used two scFv fragments containing the V_{H}6 framework which had been selected from the HuCAL (21): 2C2, binding the peptide M18 coupled to transferrin and 6B3, binding myoglobin (see Materials and Methods for details). With side-directed mutagenesis and based on our structural analysis we introduced six mutations (Q5V, S16G, T58I, V72D, S76G and S90Y) alone and in several combinations, which were hypothesized to be independently acting, individually exchangeable and were also a feature distinguishing the group of V_{H} families with favorable properties from the families with less favorable properties. We compared these mutants to the wild-type scFv fragments for effects on folding yield and, independently, the free energy of unfolding as a measure for the thermodynamic stability and determined the additivity of these mutations.

Construction of Expression Vectors

The scFv fragment 2C2 (A. Hahn et al., MorphoSys AG, unpublished results) with the human consensus domains V_{H}6 and V_{L}κ3 (H-CDR3: QRGHYGKGYKGFNSGFFDF and L-CDR3: QYYNIPT) was obtained by panning against the peptide M18 with the sequence CDAFRSEKSRQELNTIASKPPRDHVF coupled to transferrin (Jerini GmbH, Berlin), while the scFv fragment 6B3 (S. Müller et al., MorphoSys AG, unpublished results) with V_{H}6 and V_{L}λ3 (H-CDR3: SYFISFFSFDY and L-CDR3: SYDSGFSTV) was obtained by panning against myoglobin from horse skeletal muscle (Sigma). Both scFv fragments were subcloned via the restriction sites XbaI and EcoRI into the expression plasmid pMX7 (21). The different mutations were introduced with the QuikChange™ site-directed mutagenesis kit from Stratagene according to the manufacturers instructions. Multiple mutations were constructed by exchanging restriction fragments using unique XbaI, XhoI, BsaBI and EcoRI sites in the antibody. The final expression cassettes consist of a phoA signal sequence, short FLAG-tag (DYKD), the scFv fragment in the orientation V_{H}6 domain-(Gly₄Ser)₄ linker-V_{L} domain, followed by long FLAG-tag (DYKDDDD) and a hexahistidine-tag.

Expression and Purification

Thirty mL dYT medium (containing 30 μg/mL chloramphenicol, 1.0% glucose) was inoculated with a single bacterial colony and shaken overnight at 25° C. One liter of dYT medium (containing 30 μg/mL chloramphenicol, 50 mM K₂HPO₄) was inoculated with this preculture and incubated at 25° C. (5 L flask with baffles, 105 rpm). Expression was induced at an OD₅₅₀ of 1.0 by addition of IPTG to a final concentration of 0.5 mM. Incubation was continued for 18 hours while the cell density reached an OD₅₅₀ between 8.0 and 11.0. Cells were collected by centrifugation (8000 g, 10 min at 4° C.), resuspended in 40 ml of 50 mM Tris-HCl (pH 7.5) and 500 mM NaCl and disrupted by French Press lysis. The crude extract was centrifuged (48,000 g, 60 minutes at 4° C.) and the supernatant passed through a 0.2 μm filter. The proteins were purified using the two column coupled in-line procedure (4). In this strategy, the eluate of an immobilized metal ion affinity chromatography (IMAC) column, which exploits the C-terminal His-tag, was directly loaded onto an ion-exchange column. Elution from the ion-exchange column was achieved with a 0-800 mM NaCl gradient. The constructs derived from the scFv 2C2 were purified with a HS cation-exchange column in 10 mM MES (pH 6.0) and those derived from 6B3 with an HQ anion-exchange column in 10 mM Tris-HCl (pH 8.0). Pooled fractions were dialyzed against 50 mM Na-phosphate, pH 7.0, 100 mM NaCl. Protein concentrations were determined by OD₂₈₀. The soluble yield was normalized to a one liter bacterial culture with an OD₅₅₀ of 10.

Gel Filtration Chromatography

Samples of purified scFv fragments were analyzed on a Superdex-75 column equilibrated with 50 mM Na-phosphate, pH 7.0, 500 mM NaCl, on a SMART-system (Pharmacia). The samples were injected at a concentration of 5 μM in a volume of 50 μl, and the flow-rate was 60 μl/min. Lysozyme (14 kDa), carbonic anhydrase (29 kDa) and bovine serum albumin (66 kDa) were used as molecular weight standards.

Equilibrium Denaturation Experiments

Fluorescence spectra were recorded at 25° C. with a PTI Alpha Scan spectrofluorimeter (Photon Technologies, Inc., Ontario, Canada). Slit widths of 2 nm were used both for excitation and emission. Protein/GdnHCl-mixtures (1.6 ml) containing a final protein concentration of 0.5 μM and denaturant concentrations ranging from 0 to 5 M GdnHCl were prepared from freshly purified protein and a GdnHCl stock solution (8 M, in 50 mM Na-phosphate, pH 7.0, 100 mM NaCl). Each final concentration of GdnHCl was determined by measuring the refractive index. After overnight incubation at 10° C., the fluorescence emission spectra of the samples were recorded from 320 to 370 nm with an excitation wavelength of 280 nm. With increasing denaturant concentrations, the maxima of the recorded emission spectra shifted from about 340 to 350 nm. The fluorescence emission maximum was determined by fitting the fluorescence emission spectrum to a Gaussian function and was plotted versus the GdnHCl concentration. Protein stabilities were calculated as described (22, 23). To compare scFv denaturation curves in one plot the emission maxima were scaled by setting the highest value to 1 and the lowest to 0 to give normalized emission maxima.

Enzyme Linked Immunosorbent Assay (ELISA)

Myoglobin from horse skeletal muscle (Sigma) and peptide M18 coupled to transferrin (Jerini GmbH, Berlin) at a concentration of 5 μg/ml in 50 mM Na-phosphate, 100 mM NaCl, pH 7.0 were coated overnight at 4° C. on Maxisorb 96-well plates (Nunc). Plates were blocked in 2.0% sucrose, 0.1% bovine serum albumin (Sigma), 0.9% NaCl for 2 h at room temperature. After incubation of samples at concentrations from 2 μM to 0.125 μM, bound scFv fragments were detected using an α-tetra-his antibody (Qiagen) followed by an anti-mouse antibody conjugated with alkaline phosphatase (Sigma).

BIAcore Measurements

BIAcore analysis was performed using a CM5-chip (Amersham Pharmacia) with one lane coated with 2,700 resonance units (RU) of myoglobin from horse skeletal muscle (Sigma), one coated with 2,500 RU peptide M18 coupled to transferrin (Jerini GmbH, Berlin) and one blank lane as a control surface. Each binding-regeneration circle was performed at 25° C. with a constant flow rate of 25 μL/min with different antibody concentrations ranging from 5 μM to 0.08 μM in 20 mM HEPES (pH 7.0), 150 mM NaCl and 0.005% Tween 20 and 2 M NaSCN for regeneration. Determination of the antigen dissociation constant in solution was performed with competition BIAcore (24, 25) with the same chip, buffer and regeneration conditions. ScFv fragments at constant concentration and variable amounts of antigen were preincubated at least for one hour at 10° C. and injected in a sample volume of 100 μL. Data were evaluated by using BIAevaluation software (Pharmacia) and SigmaPlot (SPSS Inc.). Slopes of the association phase of linear sensograms were plotted against the corresponding total antigen concentrations and the dissociation constant was calculated as described previously (26).

Properties of the Wild Type scFv Fragments

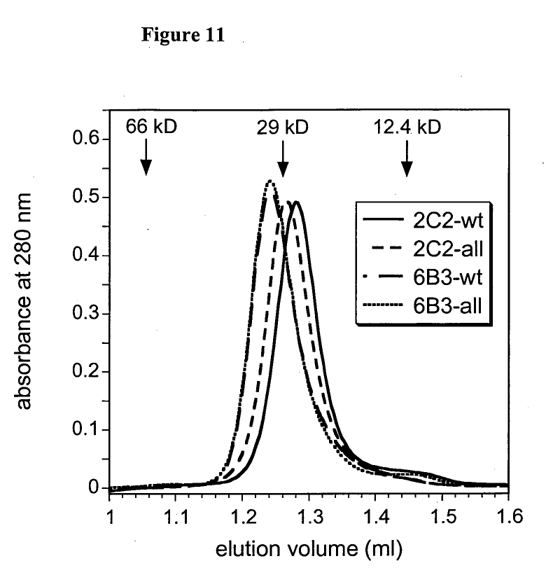

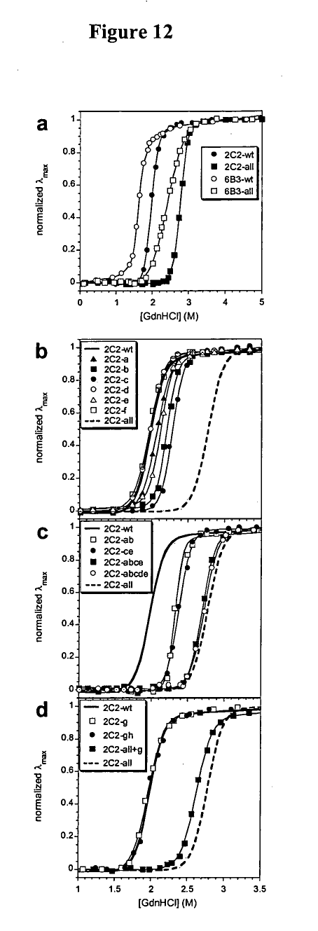

We chose the V_{H}6 framework as the model system to test our strategy for improving the biophysical properties by a structure-based design and used two scFv fragments selected from the HuCAL as model systems: 2C2, which binds the peptide M18 coupled to transferrin, and consists of V_{H}6 paired with V_{κ}3, and 6B3, which binds myoglobin, consisting of V_{H}6 paired with V_{λ}3. The two antibodies differ in CDR3 (see Materials and Methods), but otherwise the V_{H}6 sequence is identical. The wild-type (wt) scFv fragments 2C2 and 6B3 were expressed in the periplasm of E. coli. The scFv fragments were purified from the soluble fraction of the cell extract by immobilized metal affinity chromatography (IMAC), followed by an ion-exchange column. The purity of the scFv fragments was greater than 98%, as determined by SDS-PAGE (data not shown). The soluble yield after purification of a one liter bacterial culture normalized to OD₅₅₀ of 10 of 2C2-wt and 6B3-wt was 1.2±0.1 mg and 0.4±0.1 mg, respectively. Approximately 10% and 25%, respectively, of the total amount of expressed protein was found in insoluble form, as determined by Western Blot. The oligomeric state was determined by analytical gel filtration. Both proteins elute with an apparent molecular weight of 29 kDa, indicating that they are monomeric (FIG. 11). The thermodynamic stability of each protein was measured by equilibrium GdnHCl denaturation. Unfolding of the scFv fragments was monitored by the shift of the fluorescence emission maximum as a function of denaturant concentration. FIG. 12(a) shows the denaturation curve of 2C2-wt and 6B3-wt. Both curves show only one transition, indicating that V_{H} and V_{L} within the scFv fragment denature simultaneously (10). Since the fluorescence intensity of the folded and unfolded state is similar, and the maximum changes by only 17 nm, the shift in maximum can be used to determine the population of unfolded molecules (27). Under the assumption that the unfolding of the scFv fragments is a two-state process, the free energy of unfolding ΔG_{N-U} can be determined (28, 29). 2C2-wt showed a ΔG_{N-U} of 51.3 kJ/mol and 6B3-wt a ΔG_{N-U} of 51.3 kJ/mol with m-values of 25.2 kJ mol⁻¹ M⁻¹ and 27.4 kJ mol⁻¹ M⁻¹. These m-values lie in the expected range for proteins of this size indicating that both scFv fragments have the cooperativity expected for a two-state process (30).

Structural Rationale for the Selection of Mutations

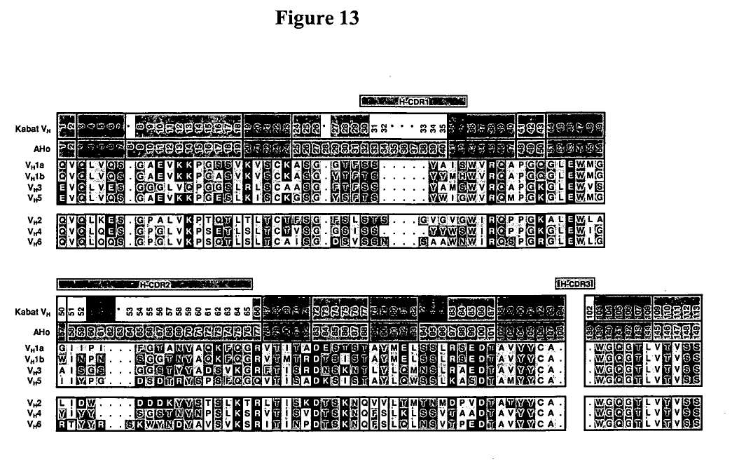

The first set of mutants to improve the properties of scFv fragments 2C2 and 6B3 containing the human V_{H}6 framework was chosen from the analysis of the structural model, guided by the sequence alignment of the human consensus V_{H} domains grouped by V_{H} domains with favorable biophysical properties (families 1, 3 and 5) and V_{H} domains with less favorable properties (families 2, 4 and 6) (FIG. 13). We focused on residues of the framework and excluded the CDR regions, since we aim to identify generically applicable mutations unlikely to affect antigen binding. The residues that we investigated in 2C2 and 6B3, together with the reasoning behind the specific changes are the following:

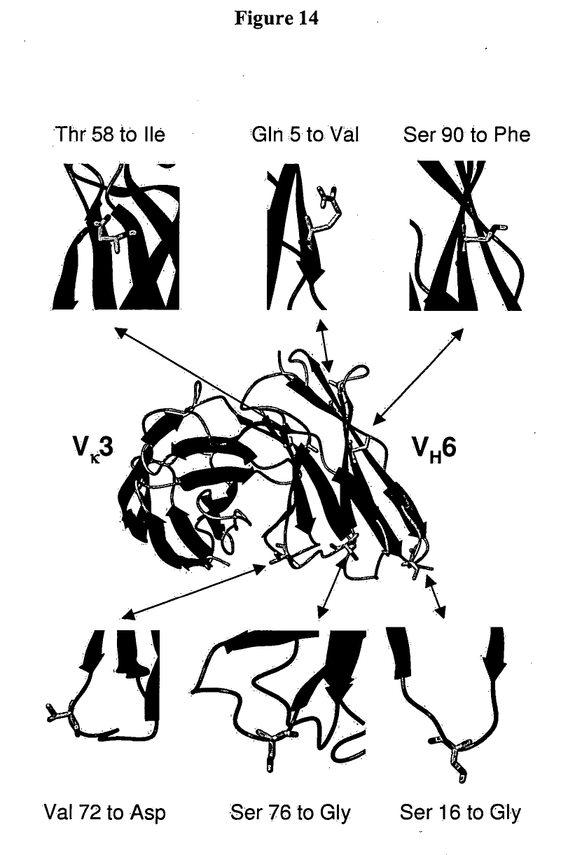

Q5V: In a selection experiment of the scFv 4D5Flu favoring stability, Val was selected at this position out of Val, Gln, Leu, and Glu (18). Position 5 is part of the first β-strand and Val has a higher β-sheet propensity as Gln (31). Nevertheless, it was shown previously that mutations of exposed hydrophobic residues have a profound effect on the in vivo folding yield (9). FIG. 14 shows that Gln in position 5 of the model of a V_{H}6-V_{L}κ3 scFv fragment (21) (PDB entries: 1DHZ (V_{H}6) and 1DH5 (V_{L}κ3)) is exposed to solvent and therefore the hydrophilic residue Gln or Lys of V_{H}2, V_{H}4 and V_{H}6 might be thought to enhance folding efficiency in contrast to the hydrophobic Val in V_{H}1a, V_{H}1b, V_{H}3, and V_{H}5. In summary, this mutation increases β-sheet propensity at the expense of creating an exposed hydrophobic residue.

S16G: V_{H}2, V_{H}4 and V_{H}6 carry a non-glycine residue, nevertheless with a conserved positive phi angle at position 16 in the loop of framework 1 (FIG. 14), which probably causes an unfavorable local conformation. Structures that have been determined with a non-Gly residue at position 16 (e.g. PDB entries 1C08, 1DQJ, 1F58) indeed show that the positive phi angle is locally maintained, apparently enforced by the surroundings. In contrast, the odd-numbered V_{H} all have Gly at this position.

T58I: The residue at position 58, which is the highly conserved Ile, points into the hydrophobic core (FIG. 14). Only V_{H}6 has Thr at this position burying an unsatisfied hydrogen bond donor. Therefore, this residue was changed to Ile.

V72D: The solvent exposed residue 72 (FIG. 14) was changed in the antibody McPC603 from Ala to Asp, which increased the ratio of protein found in the soluble periplasmic fraction compared to the insoluble periplasmic fraction 20-fold, but did not measurably alter the thermodynamic stability (15), indicating hat it might have an effect on the folding efficiency. Only the consensus sequence of the most stable V_{H} family V_{H}3 has Asp at this position.

S76G: The odd numbered V_{H} domains have Gly at position 76 in framework 2 (FIG. 14) in contrast to the even numbered V_{H} domains, which carry Thr or Ser. In half of the known antibody structures found in the PDB, the residue at this position has a positive phi angle, indicating that glycine could be a better choice at this position.

S90Y: The semi-buried position 90 (FIG. 14) of V_{H}1a, V_{H}1b, V_{H}3, and V_{H}5 is occupied by Tyr, whereas V_{H}2, V_{H}4, and V_{H}6 have Val or Ser. This residue is part of the β-sheet of the immunoglobulin fold and is exchanged to Ser in V_{H}6, but Tyr has a higher β-sheet propensity than Ser (31).

In position 20 and 88 group-specific differences are seen, too (FIG. 13). The residues in both positions are solvent exposed and participate in a β-sheet. At position 20 the odd-numbered V_{H} domains have the basic residues Lys and Arg, while the even-numbered domains show Thr or Ser. In position 88 all domains with favorable properties contain Thr and the domains with unfavorable properties contain Gln. However, as all theses residues are hydrophilic and have similar β-sheet propensities, it might be expected that the differences in folding efficiency is small. Therefore, these residues were not exchanged.

Single Mutations

The six mutations (Q6V, S16G, T58I, V72D, S76G agfnd S90Y) described above were introduced into 2C2-wt and 6B3-wt by site directed mutagenesis. All scFv fragments carrying one mutation were expressed and purified in an identical manner to the wild type scFv fragments and were monomeric in solution (data not shown). In all single and subsequently constructed multiple mutants the proportion of soluble to insoluble protein in the periplasm stayed constant, even in those cases where the total expression level increased. The biophysical data are summarized in Table 7 To compare the improvements caused by the mutations in 2C2 and 6B3, the expression yield of soluble protein is normalized to the yield of the corresponding wild-type scFv fragments and the free energy of unfolding (ΔG_{N-U}) is given as the difference (ΔΔG_{N-U}) to the corresponding scFv-wt. The denaturant-induced unfolding curves are shown in FIG. 12(b).

Both single mutations exchanging the non-gycine residues with positive phi-angles (S16G and S76G) increased the yield of soluble protein by a factor of approximately two. The thermodynamic stability was also increased in both single mutations with ΔΔG_{N-U} of 6.2 and 7.3 kJ/mol for 2C2-S16G and 6B3-S16G and ΔΔG_{N-U} of 3.7 and 3.5 kJ/mol for 2C2-S76G and 6B3-S76G, respectively, compared to the wild-type scFv fragments. The mutation to Gly in a loop region causes a higher flexibility, which enables the optimal orientation of the anti-parallel β-sheet stabilizing the whole domain. The higher yield of these mutants is probably due to the increased protease resistance and folding efficiency caused by the stabilized folded state of the protein.

The mutation of the OH-carrying Thr58 to Ile, pointing into the hydrophobic core, did not alter the yield of soluble protein but caused a marked increase of thermodynamic stability with ΔΔG_{N-U} of 7.9 and 6.8 kJ/mol for 2C2-T58I and 6B3-T58I, respectively. This remarkable improvement in stability is due to the additional van der Waals interaction of the hydrophobic Ile within the hydrophobic core and to the absence of the desolvation necessary when burying Thr. Interestingly, this mutation does not have an effect on the yield of soluble protein, indicating that the folding efficiency is not increased.

Both mutations exchanging a residue in a β-sheet to a residue with higher β-sheet propensity (Q5V and S90Y) resulted in an approximately 1.8-fold increase in yield of soluble protein. In addition, the thermodynamic stability is slightly increased with the exception of 2C2-S90Y, which shows even a very small decrease in comparison to the wild-type scFv fragment. The analysis of these constructs shows that mutations of residues, which participate in a β-sheet, to a residue with higher β-sheet building propensity can increase yield of soluble protein due to a higher folding efficiency. Depending on the scFv fragment the thermodynamic stability is also increased probably because of better orientation of the mutated residue, facilitating the orientation of stabilizing hydrogen bonds in the β-sheet.

The last single mutation exchanges a solvent-exposed hydrophobic residue with a hydrophilic one (V72D). The yield of soluble protein in 2C2-V72D and 6B3-V72D is increased 3.2 and 1.8 fold, respectively. The thermodynamic stability in 2C2-V72D is not changed, while in 6B3-V72D it is slightly increased with ΔΔG_{N-U} of 2.2 kJ/mol.

Multiple Mutations

To determine whether the improvements were additive, we cloned combinations of the single mutations. The scFv fragments with multiple mutations were expressed and purified as above and were also monomeric in solution, as demonstrated by analytical gel filtration (2C2- and 6B3-all as examples in FIG. 11). The denaturation curves of all multiple mutants of 2C2 tested showed one steep, cooperative transition (FIG. 12(d)), indicating that the V_{κ}3 domain is also stabilized with the help of the six mutations in V_{H}6, probably because the mutated V_{H}6 domain stabilizes V_{κ}3 through the hydrophobic V_{H}-V_{L} interface interactions. In contrast, the transition of the equilibrium unfolding of the double mutants 6B3-Q5V+S16G and 6B3-T58I+S76G revealed a lower cooperativity compared to 6B3-wt and gave m-values of 18.9 and 19.3 kJ mol⁻¹ M⁻¹, respectively, indicating that the unfolding is no longer a two-state process. The scFv fragment 6B3 carrying all six mutations derived from the sequence comparison with the group of V_{H} domains with favorable properties (6B3-all) showed an even lower cooperativity and has an m-value of 14.3 kJ mol⁻¹ M⁻¹ (FIG. 12(a)). The V_{λ}3 domain, which has the lowest thermodynamic stability of isolated V_{L} domains (see Example 1, 11), probably starts to unfold first in the scFv 6B3 with multiple mutations, while the mutated, stabilized V_{H}6 domain is still folded and only unfolds at higher concentrations of denaturant. Because of this lack of 2-state behavior, the ΔG_{N-U} values could not be calculated for the multiple mutants of 6B3.

The details of the yield of soluble protein and thermodynamic stability determinations are listed in Table 7. In summary, the effect on yield and stability of the single mutations is almost fully additive. The scFv fragments carrying all six mutations, 2C2-all and 6B3-all, show an increase in yield of 4.3 and 4.2 fold, respectively, compared to the wild-type scFv fragments. The absolute values for 2C2-all are a yield of 5.1 mg/L, which is 3.9 mg/L more than for 2C2-wt, and a thermodynamic stability of 72.3 kJ/mol. In the case of 6B3-all, a yield of 1.7 mg/L was obtained, which is 1.3 mg/L more than for 6B3-wt.

Analysis of Framework 1 Subtype

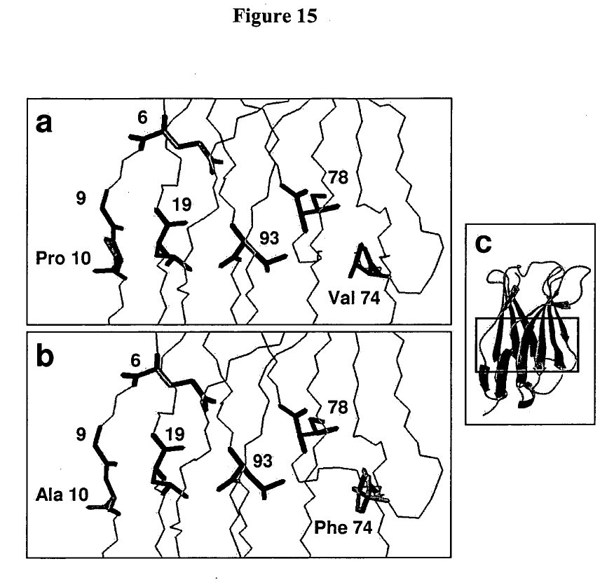

V_{H} structures can be divided into four distinct framework 1 conformations depending on the type of amino acids at position 6, 7 and 10 (32) (numbering scheme is according to Honegger & Plückthun (33)). Residues at position 19, 74, 78 and 93, which are part of the hydrophobic core of the lower part of the domain and thus influence thermodynamic stability and folding efficiency, are correlated to this structural subtype (32). While the V_{H} domains with the most favorable properties fall into subtype II (V_{H}3) and subtype III (V_{H}1a, V_{H}1b and V_{H}5), the V_{H} domains with less favorable properties V_{H}2 and V_{H}4 fall into subgroup I. V_{H}6, which we want to improve, can be assigned to subtype III which is defined by Gln at position 6 and the absence of Pro at position 7 (32). Analysis of subtype III defining and correlated residues of human V_{H} domains (32) shows that the V_{H}6 fragment carries rarely used residues in position 10, 74 and 78 (Table 8). Pro in position 10 is used in 8% of the sequences, whereas Ala is used in 76% of the sequences. Pro only allows a more limited number of conformations than Ala. In a mutagenesis experiment (34), Pro at position 10 was shown to destabilize a V_{H} domain in a subtype IV context (only occurring in murine, not in human sequences). Val at position 74 and Ile at position 78 have a frequency of 1% and 8%, respectively, compared to V_{H} subtype III sequences. Val74 was exchanged in 2C2 and 6B3 to the more frequently found Phe, as the bulky aromatic amino acid probably increases the packing density of the hydrophobic core. Ile78 was not exchanged to the subtype III consensus residues Ala or Val, which are, as Ile, non-aromatic aliphatic residues, as the effect on the packing density would probably be small. In FIG. 15(a) the framework 1 subtype determining and correlated residues are shown in the model of V_{H}6 (21) (PDB entry: 1DHZ), and in FIG. 15(b) the model of the double mutation is shown with P10A (Pro to Ala at position 10) and V74F.

The mutations to the framework 1 subtype III consensus P10A alone and in combination with V74F were introduced into the wild-type scFv fragments by site directed mutagenesis. 2C2-P10A and 6B3-P10A showed a 2.9 and 4.2 fold increase in yield of soluble protein compared to the wild-type scFv fragments, respectively, while the double mutants with P10A and V74F showed a lower increase with 1.9 and 1.7 fold, respectively. All biophysical data are summarized in Table 7. The analysis of the soluble and insoluble fraction of the periplasmic expression in E. coli of the single- and double-mutant showed that both the total expression level and the level of soluble protein increased by the mutations and thus the ratio between soluble and insoluble scFv fragment remained constant (data not shown). The thermodynamic stability of the scFv fragments 2C2 and 6B3 is not increased by the mutation P10A, and is only slightly increased (ΔΔG_{N-U} of 0.5 kJ/mol and 0.4 kJ/mol, respectively) with the double-mutation P10A and V74F (Table 7, FIG. 12(d)). The biophysical analysis therefore shows that the mutation P10A indeed increases the folding efficiency, as demonstrated by the higher yield of periplasmic protein but did not change stability in comparison to the wild-type scFv fragments. In contrast, the mutation V74F may slightly increase the stability because of enhanced stabilizing interactions in the hydrophobic core, probably at the expense of folding efficiency, since the positive effect of P10A on yield is decreased in the double-mutant. Because of the higher yield of the single-mutant P10A compared to the double-mutant P10A+V74F, which showed only a small increase in thermodynamic stability, we cloned only the mutation P10A into 2C2-all and 6B3-all, resulting in the construct scFv-all+P10A. The yields compared to 2C2-all and 6B3-all were decreased 0.8 and 2.1 fold, respectively. In the case of 2C2-all+P10A the thermodynamic stability with ΔG_{N-U} of 68.1 kJ/mol was 4.1 kJ/mol lower than the stability of 2C2-all. The midpoint of denaturation, which is a semi-quantitative measure for the thermodynamic stability, in 6B3-all+P10A was also at lower GdnHCl concentration than the midpoint of 6B3-all.

Determination of Binding Activity

The goal of the study was to show that yield and stability of V_{H}6 containing scFv fragments can be improved by the structure-based approach, guided by the family-specific analysis, while the binding activity is retained. We analyzed the binding activity with two independent methods: ELISA and BIAcore. For the ELISA, we coated the corresponding antigen and applied various concentrations of scFv fragments. We tested all single mutations including scFv-P10A and the multiple mutations scFv-all and scFv-all+P10A. All mutants show similar concentration dependence, which indicates that they have the same binding affinity (data not shown).

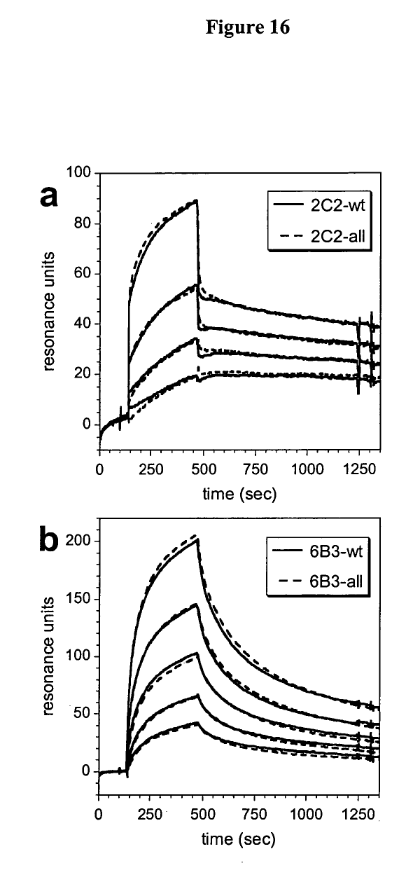

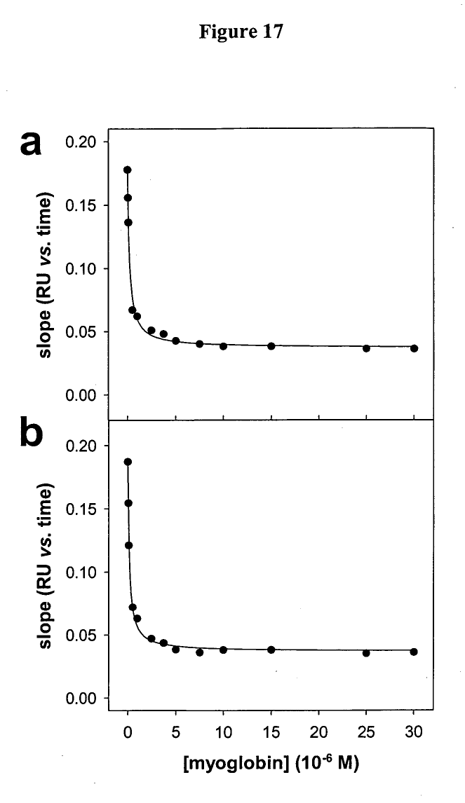

BIAcore experiments were performed with different concentrations of scFv fragments flowing over an antigen-coated chip. FIGS. 16a and 16b show an overlay of 2C2-wt and -all and 6B3-wt and -all, respectively, plotted as resonance units (RU) vs. time. The association and dissociation curves of scFv-wt and -all to the antigen-coated chip superpose in both cases, indicating that the binding is fully retained. However, the dissociation phase did not reach the background level before injection of scFv fragments, preventing unambiguous determination of the antigen dissociation constant (K_{d}). This unspecific binding was observed at different antigen-coating densities (2,700 RU and 370 RU, data not shown). This indicates that this behavior is not due to rebinding on the chip but may be due to a small portion partially unfolded scFv fragment that sticks nonspecifically to the antigen-coated chip. Therefore, competition BIAcore experiments (24, 25) were performed to determine K_{d} in solution. In this experiment, scFv protein was incubated with soluble antigen, and the mixture was injected on a BIAcore chip containing immobilized antigen. Only free scFv, but not antigen-bound scFv, could bind to antigen on the surface. Thereby, the dissociation constant in solution can e determined, independent of any unspecific binding events. From the previous experiments K_{d} was estimated to be around 10⁻⁷ M. Therefore, competition BIAcore experiments were performed with 6B3-wt and 6B3-all at 16 nM and 10 nM, respectively, in the presence of different concentrations of myoglobin ranging from 50 nM to 30 μM. From a plot of the slope of the association phase against the corresponding total antigen concentration in solution, K_{d} of 6B3-wt was calculated as (1.9±0.5)·10⁻⁷ M and that of 6B3-all as (1.5±0.4)·10⁻⁷ M as described previously (26) (FIG. 17). Both K_{d} values lie in the experimental error range indicating that the binding is fully retained.

The aim of this study was to demonstrate the validity of the structure-based, family-consensus based predictions. We chose scFv fragments containing the human germline family V_{H}6 consensus domain as a model system to improve the expression yield of soluble protein and thermodynamic stability. Potential mutations improving these biophysical properties were identified from comparison of the residues which define the framework 1 subtype and other interacting residues to the consensus found within the same subtype. The next set of potential mutations was found by an analysis of the structure for potential imperfections, guided by a comparison to the consensus sequences of those V_{H} domains with known favorable biophysical properties (families 1, 3 and 5). We excluded CDR residues from this analysis. We could pinpoint such residues, as we had previously systematically determined the biophysical properties of consensus sequences of all human variable domain subgroups (see Example 1, 11). The experiment shows that all seven proposed single mutations fall into three categories. They result either only in an increase in expression yield of soluble protein, or only in thermodynamic stability, or both. This distinction helps to understand the role of these residues in determining the biophysical properties of this proteins. In case of the scFv 2C2 three and in case of the scFv 6B3 even five out of these seven mutations result in an improvement of both biophysical properties. These results illustrate that the combination of structure-based analysis, guided by family alignments, is a powerful way to improve the properties of immunoglobulin variable domains. Since our analysis (see Example 1, 11) covers all human families, we have now a general strategy for this task.

The analysis of different combinations of the single mutations to the consensus of V_{H} domains with favorable properties showed that the improvements in free energy were almost perfectly additive, indicating that they act independently. The mutant with the highest yield and thermodynamic stability compared to the wild-type scFv fragments is indeed the mutant with all six mutations. In the case of the scFv 2C2, the properties of the best mutant are comparable to the properties of a model scFv fragment consisting of the most stable V_{H} domain, V_{H}3, and the same V_{L} domain V_{κ}3 with a different CDR3, which was part of the systematic biophysical characterization of human variable antibody domains (see Example 1, 11), indicating that it is indeed possible to turn an antibody with unfavorable properties into a one with very favorable properties by changing only a few residues. Most importantly, both CDRs and those framework residues are maintained which are important for binding.

The addition of the mutation P10A to the scFv fragments carrying six mutations decreases both expression yield and thermodynamic stability, although in the wild-type scFv fragments this mutation increased the soluble yield 2.9-fold in the case of 2C2-P10A and 4.2-fold in the case of 6B3-P10A and left the thermodynamic stability unchanged. The mutations Q5V and S16G, which are close to position 10, should still be beneficial to the V_{H}6 framework as they are independent of the type of amino acid in position 10. The reason of the declined biophysical properties of this mutation in the context of the improved framework can probably only be explained with the help of the experimentally determined 3D structure.

The improvements seem to be independent of the V_{L} domain and of the sequence and length of CDR3, as 2C2 with V_{κ}3 and 6B3 with V_{λ}3 and different H-CDR3 loops gave similar results. There were only two minor exceptions, as the thermodynamic stability of the 6B3 mutants V72D and S90Y is slightly increased, while in 2C2 no stability increase could be observed. It was shown previously that in scFv fragments V_{λ} domains, in contrast to V_{κ} domains, are able to form very stable V_{H}-V_{L} interfaces, increasing the stability of the whole scFv fragment even above the intrinsic stabilities of the isolated domains (see Example 1, 11). The residue at position 72 is not involved in the interface interactions but is in close proximity to it (FIG. 14). It is therefore possible that the mutation V72D may lead to a small change in the orientation of the interface, which has no effect on V_{κ}3 domains in 2C2 but a small stabilizing effect through the interface interactions with the V_{λ}3 domain of 6B3. The residue in position 90 is on the side opposite to the interface to V_{L} (FIG. 14) and also 29 residues away from the CDR3 indicating that the slightly increased stability of 6B3 is probably not due to the different V_{L} domain and CDR3 sequences compared to 2C2.

Although we did not exchange residues of the CDR with possible direct contact to the antigen, it could not be a priori excluded that changes in the framework might affect the orientation of the CDRs and, thereby, antigen binding. Therefore, we experimentally determined the binding properties. However, in the case of the examined mutations, antigen binding was fully retained as demonstrated by three independent methods.

In this study we show that it is possible to rationally transform antibody frameworks with less favorable properties into those with very favorable properties while retaining their binding activity and the binding characteristics of the framework. It could be argued that an easier approach would be to use directly the very stable V_{H}3 framework with a suitable V_{L} domain. Nevertheless, framework residues can affect the orientation of CDRs, can be part of the hapten-binding cavity located in the V_{H}-V_{L} interface and build the “outer loop”, which was seen in some cases to be involved in antigen binding. These “framework” residues can thereby contribute greatly to affinity and diversity and it is unlikely that a single framework can provide the ideal solution in all cases. Therefore, we believe that the preferred approach to achieve a structurally diverse library of stable frameworks is to optimize the human consensus antibody frameworks further in the way we presented here, as it would give access to a whole range of stable scaffolds covering all natural families.

In this study we focused on the improvement of the V_{H}6 framework. However, because of the sequence similarity five of the mutations studied (Q5V, S16G, V72D, S76G and S90Y) should give similar results for V_{H} domains belonging to family V_{H}2 and V_{H}4. While this approach is useful for the design of antibody libraries, in many cases given human antibodies, e.g. from transgenic mice (35, 36), obtained by humanization (37) or by phage display from a library of natural sequences (38-40) may also benefit from improvement.

These results also show that some human germline genes do not encode an optimal version of the protein, regarding its biophysical properties. Since the biophysical properties of natural domains cover a wide range, it cannot be argued that limited stability is a desirable property for the immune system. Rather, the stability of V_{H}2, V_{H}4 and V_{H}6 may simply be good enough to be tolerated by the immune system. For those biomedical or biotechnological applications where it is not good enough, however, we have now provided a pathway to improve these properties in a straightforward way.

### References for Example 2

1. Bird, R. E., Hardman, K. D., Jacobson, J. W., Johnson, S., Kaufman, B. M., Lee, S. M., Lee, T., Pope, S. H., Riordan, G. S., and Whitlow, M. (1988) Single-chain antigen-binding proteins, Science 242, 423-426.
2. Glockshuber, R., Malia, M., Pfitzinger, I., and Plückthun, A. (1990) A comparison of strategies to stabilize immunoglobulin Fv-fragments, Biochemistry 29, 1362-1367.
3. Huston, J. S., Levinson, D., Mudgett-Hunter, M., Tai, M. S., Novotny, J., Margolies, M. N., Ridge, R. J., Bruccoleri, R. E., Haber, E., Crea, R., and et al. (1988) Protein engineering of antibody binding sites: recovery of specific activity in an anti-digoxin single-chain Fv analogue produced in Escherichia coli, Proc. Natl. Acad. Sci. USA 85, 5879-5883.
4. Plückthun, A., Krebber, A., Horn, U., Knüpfer, U., Wenderoth, R., Nieba, L., Proba, K., and Riesenberg, D (1996) in Antibody Engineering, A Practical Approach (Mc Cafferty, J., Hoogenboom, H. R., and Chiswell, D. J., eds), pp. 203-252, Oxford University Press, New York
5. Shusta, E. V., Raines, R. T., Plückthun, A., and Wittrup, K. D. (1998) Increasing the secretory capacity of Saccharomyces cerevisiae for production of single-chain antibody fragments, Nat. Biotechnol. 16, 773-777.
6. Rees, A. R., Staunton, D., Webster, D. M., Searle, S. J., Henry, A. H., and Pedersen, J. T. (1994) Antibody design: beyond the natural limits, Trends Biotechnol. 12, 199-206.
7. Wörn, A., and Plückthun, A. (2001) Stability engineering of antibody single-chain Fv fragments, J. Mol. Biol. 305, 989-1010.
8. Bucciantini, M., Giannoni, E., Chiti, F., Baroni, F., Formigli, L., Zurdo, J., Taddei, N., Ramponi, G., Dobson, C. M., and Stefani, M. (2002) Inherent toxicity of aggregates implies a common mechanism for protein misfolding diseases, Nature 416, 507-511.
9. Nieba, L., Honegger, A., Krebber, C., and Plückthun, A. (1997) Disrupting the hydrophobic patches at the antibody variable/constant domain interface: improved in vivo folding and physical characterization of an engineered scFv fragment, Protein Eng. 10, 435-444.
10. Wörn, A., and Plückthun, A. (1999) Different equilibrium stability behavior of ScFv fragments: identification, classification, and improvement by protein engineering, Biochemistry 38, 8739-8750.
11. Ewert, S., Huber, T., Honegger, A., and Plückthun, A. (2002) Biophysical properties of human variable antibody domains, JMB, submitted
12. Bothmann, H., and Plückthun, A. (1998) Selection for a periplasmic factor improving phage display and functional periplasmic expression, Nat. Biotechnol. 16, 376-380.
13. Bothmann, H., and Plückthun, A. (2000) The periplasmic Escherichia coli peptidylprolyl cis,trans-isomerase FkpA. I. Increased functional expression of antibody fragments with and without cis-prolines, J. Biol. Chem. 275, 17100-17105.
14. Kipriyanov, S. M., Moldenhauer, G., Martin, A. C., Kupriyanova, O. A., and Little, M. (1997) Two amino acid mutations in an anti-human CD3 single chain Fv antibody fragment that affect the yield on bacterial secretion but not the affinity, Protein Eng. 10, 445-453.
15. Knappik, A., and Plückthun, A. (1995) Engineered turns of a recombinant antibody improve its in vivo folding, Protein Eng. 8, 81-89.
16. Forsberg, G., Forsgren, M., Jaki, M., Norin, M., Sterky, C., Enhorning, A., Larsson, K., Ericsson, M., and Björk, P. (1997) Identification of framework residues in a secreted recombinant antibody fragment that control production level and localization in Escherichia coli, J. Biol. Chem. 272, 12430-12436.
17. Sieber, V., Plückthun, A., and Schmid, F. X. (1998) Selecting proteins with improved stability by a phage-based method, Nat. Biotechnol. 16, 955-960.
18. Jung, S., Honegger, A., and Plückthun, A. (1999) Selection for improved protein stability by phage display, J. Mol. Biol. 294, 163-180.
19. Jermutus, L., Honegger, A., Schwesinger, F., Hanes, J., and Pluckthun, A. (2001) Tailoring in vitro evolution for protein affinity or stability, Proc. Natl. Acad. Sci. USA 98, 75-80.
20. Steipe, B., Schiller, B., Plückthun, A., and Steinbacher, S. (1994) Sequence statistics reliably predict stabilizing mutations in a protein domain, J. Mol. Biol. 240, 188-192.
21. Knappik, A., Ge, L., Honegger, A., Pack, P., Fischer, M., Wellnhofer, G., Hoess, A., Wölle, J., Plückthun, A., and Virnekäs, B. (2000) Fully synthetic human combinatorial antibody libraries (HuCAL) based on modular consensus frameworks and CDRs randomized with trinucleotides, J. Mol. Biol. 296, 57-86.
22. Pace, C. N., and Scholtz, J. M. (1997) in Protein Structure, A Practical Approach (Creighton, ed), pp. 299-321, Oxford University Press, New York
23. Jäger, M., Gehrig, P., and Plückthun, A. (2001) The scFv fragment of the antibody hu4D5-8: evidence for early premature domain interaction in refolding, J. Mol. Biol. 305, 1111-1129.
24. Karlsson, R. (1994) Real-time competitive kinetic analysis of interactions between low-molecular-weight ligands in solution and surface-immobilized receptors, Anal. Biochem. 221, 142-151.
25. Nieba, L., Krebber, A., and Plückthun, A. (1996) Competition BIAcore for measuring true affinities: large differences from values determined from binding kinetics, Anal. Biochem. 234, 155-165.
26. Hanes, J., Jermutus, L., Weber-Bornhauser, S., Bosshard, H. R., and Plückthun, A. (1998) Ribosome display efficiently selects and evolves high-affinity antibodies in vitro from immune libraries, Proc. Natl. Acad. Sci. USA 95, 14130-14135.
27. Eftink, M. R. (1994) The use of fluorescence methods to monitor unfolding transitions in proteins, Biophys. J. 66, 482-501.
28. Santoro, M. M., and Bolen, D. W. (1988) Unfolding free energy changes determined by the linear extrapolation method. 1. Unfolding of phenylmethanesulfonyl alpha-chymotrypsin using different denaturants, Biochemistry 27, 8063-8068.
29. Jäger, M., and Plückthun, A. (1999) Domain interactions in antibody Fv and scFv fragments: effects on unfolding kinetics and equilibria, FEBS Lett. 462, 307-312.
30. Myers, J. K., Pace, C. N., and Scholtz, J. M. (1995) Denaturant m values and heat capacity changes: relation to changes in accessible surface areas of protein unfolding, Protein Sci. 4, 2138-2148.
31. Zhu, Z. Y., and Blundell, T. L. (1996) The use of amino acid patterns of classified helices and strands in secondary structure prediction, J. Mol. Biol. 260, 261-276.
32. Honegger, A., and Plückthun, A. (2001) The influence of the buried glutamine or glutamate residue in position 6 on the structure of immunoglobulin variable domains, J. Mol. Biol. 309, 687-699.
33. Honegger, A., and Plückthun, A. (2001) Yet another numbering scheme for immunoglobulin variable domains: An automatic modeling and analysis tool, J. Mol. Biol. 309, 657-670.
34. Jung, S., Spinelli, S., Schimmele, B., Honegger, A., Pugliese, L., Cambillau, C., and Plückthun, A. (2001) The importance of framework residues H6, H7 and H10 in antibody heavy chains: experimental evidence for a new structural subclassification of antibody VH domain, J. Mol. Biol. 309, 701-716.
35. Fishwild, D. M., O'Donnell, S. L., Bengoechea, T., Hudson, D. V., Harding, F., Bernhard, S. L., Jones, D., Kay, R. M., Higgins, K. M., Schramm, S. R., and Lonberg, N. (1996) High-avidity human IgG kappa monoclonal antibodies from a novel strain of minilocus transgenic mice, Nat. Biotechnol. 14, 845-851.
36. Mendez, M. J., Green, L. L., Corvalan, J. R., Jia, X. C., Maynard-Currie, C. E., Yang, X. D., Gallo, M. L., Louie, D. M., Lee, D. V., Erickson, K. L., Luna, J., Roy, C. M., Abderrahim, H., Kirschenbaum, F., Noguchi, M., Smith, D. H., Fukushima, A., Hales, J. F., Klapholz, S., Finer, M. H., Davis, C. G., Zsebo, K. M., and Jakobovits, A. (1997) Functional transplant of megabase human immunoglobulin loci recapitulates human antibody response in mice, Nat. Genet. 15, 146-156.
37. Winter, G., and Harris, W. J. (1993) Humanized antibodies, Trends Pharmacol. Sci. 14, 139-143.
38. Hoogenboom, H. R., and Winter, G. (1992) By-passing immunisation. Human antibodies from synthetic repertoires of germline VH gene segments rearranged in vitro, J. Mol. Biol. 227, 381-388.
39. Griffiths, A. D., Williams, S. C., Hartley, O., Tomlinson, I. M., Waterhouse, P., Crosby, W. L., Kontermann, R. E., Jones, P. T., Low, N. M., Allison, T. J., Prospero, T. D., Hoogenboom, H. R., Nissim, A., Cox, J. P. L., Harrison, J. L., Zaccolo, M., Gherardi, E., and Winter, G. (1994) Isolation of high affinity human antibodies directly from large synthetic repertoires, EMBO J. 13, 3245-3260.
40. Vaughan, T. J., Williams, A. J., Pritchard, K., Osbourn, J. K., Pope, A. R., Earnshaw, J. C., McCafferty, J., Hodits, R. A., Wilton, J., and Johnson, K. S. (1996) Human antibodies with sub-nanomolar affinities isolated from a large non-immunized phage display library, Nat. Biotechnol. 14, 309-314.
41. Kabat, E. A., Wu, T. T., Perry, H. M., Gottesmann, K. S., and Foeller, C. (1991) in Sequences of Proteins of Immunological Interest, NIH Publication No. 91-3242, National Technical Information Service (NTIS)

42. Koradi, R., Billeter, M., and Wüthrich, K. (1996) MOLMOL: a program for display and analysis of macromolecular structures, J. Mol. Graph. 14, 51-55, 29-32.

TABLE 1
Summary of biophysical characterization of isolated V_{H} and V_{L} domains

| | | | soluble yield | oligo- | midpoint | | m |
|---|---|---|---|---|---|---|---|
| human | | | (mg/ L) | meric | [GdnHCl] | ΔG_{N-U} | (kJ M⁻¹ |
| family | | CDR3 | OD₅₅₀ = 10) | state | (M) | (kJ mol⁻¹) | mol⁻¹) |
| V_{H} | 1a | long^{b} | 1.0 | M^{g} | 1.5 | 13.7 | 10.1 |
| | 1b | long | 1.2 | M | 2.1 | 26.0 | 12.7 |
| | 2 | long | ref^{f} | n.d.^{h} | 1.6 | n.d. | n.d. |
| | 3 | long | 2.4 | M | 3.0 | 52.7 | 17.6 |
| | 3^{a} | short^{c} | 2.1 | n.d. | 2.7 | 39.7 | 14.6 |
| | 4 | long | ref | n.d. | 1.8 | n.d. | n.d. |
| | 5 | long | ref | M | 2.2 | 16.5 | 7.0 |
| | 6 | long | ref | n.d. | 0.8 | n.d. | n.d. |
| V_{L} | κ1 | κ-like^{d} | 4.5 | M | 2.1 | 29.0 | 14.1 |
| | κ2 | κ-like | 14.2 | M | 1.5 | 24.8 | 16.1 |
| | κ3 | κ-like | 17.1 | M | 2.3 | 34.5 | 14.8 |
| | κ4 | κ-like | 9.6 | D, Mⁱ | 1.5 | n.d. | n.d. |
| | λ1 | λ-like^{e} | 0.3 | M | 2.1 | 23.7 | 11.1 |
| | λ2 | λ-like | 1.9 | M | 1.0 | 16.0 | 16.2 |
| | λ3 | λ-like | 0.8 | D, M | 0.9 | 15.1 | 15.9 |

^{a}data from Ewert et al., 2002
^{b}long CDR3, sequence: YNHEADMLIRNWLYSDV
^{c}short CDR3, sequence: WGGDGFYAMDY
^{d}κ-like CDR3, sequence: QQHYTTPPT
^{e}λ-like CDR3, sequence: QSYDSSLSGVV
^{f}no soluble protein obtained, purification via refolding of inclusion bodies.
^{g}monomer in 50 mM sodium-phosphate (pH 7.0) and 500 mM NaCl, in case of V_{H}1a with 0.9 M GdnHCl
^{h}not determined
ⁱdimer and monomer equilibrium

TABLE 2

Sequence alignment of the human consensus V_{H} and V_{L} domains
at regions possibly influencing thermodynamic stability

| | | |
|---|---|---|
| charge cluster | upper core | lower core |

| AHo^{a} | 45 | 53 | 77 | 97 | 99 | 100 | 2 | 4 | 25 | 29 | 31 | 41 | 80 | 82 | 89 | 108 | 19 | 74 | 78 | 93 | 104 |
|---|---|---|---|---|---|---|---|---|---|---|---|---|---|---|---|---|---|---|---|---|---|
| V_{H}3 | R | E | R | R | E | D | V | L | A | F | F | M | I | R | L | R | L | V | F | M | Y |
| V_{H1}a | R | E | R | R | E | D | V | L | A | G | F | I | I | A | A | R | V | F | V | L | Y |
| V_{H1}b | R | E | R | R | E | D | V | L | A | Y | F | M | M | R | A | R | L | F | V | L | Y |
| V_{H}5 | R | E | Q | K | S | D | V | L | G | Y | F | I | I | A | A | R | L | F | V | W | Y |
| V_{H}2 | R | E | R | D | V | D | V | L | F | F | L | V | I | K | V | R | L | L | L | M | Y |
| V_{H}4 | R | E | R | T | A | D | V | L | V | G | I | F | I | V | F | R | L | L | V | L | Y |
| V_{H}6 | R | E | R | T | E | D | V | L | I | D | V | F | I | P | F | R | L | V | I | L | Y |
| V_{κ}1 | Q | K | R | Q | E | D | I | M | A | Q | I | L | G | G | F | Q | V | V | F | I | Y |
| V_{κ}2 | L | Q | R | E | E | D | I | M | S | Q | L | L | G | G | F | Q | A | V | F | I | Y |
| V_{κ}3 | Q | R | R | E | E | D | I | L | A | Q | V | L | G | G | F | Q | A | V | F | I | Y |
| V_{κ}4 | Q | K | R | Q | E | D | I | M | S | Q | V | L | G | G | F | Q | A | V | F | I | Y |
| V_{λ}1 | Q | K | R | Q | E | D | I | L | G | S | I | V | G | K | A | Q | V | V | F | I | Y |
| V_{λ}2 | Q | K | R | Q | E | D | I | L | G | S | V | V | G | K | A | Q | I | V | F | I | Y |
| V_{λ}3 | Q | V | R | Q | E | D | I | L | G | — | L | A | G | N | A | Q | A | I | F | I | Y |

^{a}Numbering according to the structurally based scheme of Honegger & Plückthun (2001)

TABLE 3

Key residues of the human V_{H} family consensus sequences

| | |
|---|---|
| | residues differing |
| residues defining | between well and poorly |
| framework I class | behaved V_{H} domains |

| | | | | | | | | | | |
|---|---|---|---|---|---|---|---|---|---|---|
| AHo^{a} | Class | 6 | 7 | 10 | 5 | 16 | 47 | 58 | 76 | 90 |

| | | | | | | | | | | |
|---|---|---|---|---|---|---|---|---|---|---|
| V_{H}3 | II | E | S | G | V | G | A | I | G | Y |
| V_{H}1a | III | Q | S | A | V | G | A | I | G | Y |
| V_{H}1b | III | Q | S | A | V | G | A | I | G | Y |
| V_{H}5 | III | Q | S | A | V | G | M | I | G | Y |
| V_{H}2 | I | E | S | P | K | T | P | I | T | V |
| V_{H}4 | I | E | S | P | Q | S | P | I | S | S |
| V_{H}6 | III | Q | S | P | Q | S | S | T | S | S |

^{a}Numbering according to the structurally based scheme of Honegger & Plückthun (2001)

TABLE 4

Sequence alignment of the human consensus V_{L}
families

| | | | | | | |
|---|---|---|---|---|---|---|
| AHo^{a} | 12 | 18 | 138 | 146 | 148 | 149 |

| | | | | | | |
|---|---|---|---|---|---|---|
| V_{κ}1 | S | R | T | E | K | R |
| V_{κ}2 | P | P | T | E | K | R |
| V_{κ}3 | S | R | T | E | K | R |
| V_{κ}4 | A | R | T | E | K | R |
| V_{κ}1 | S | R | V | T | L | G |
| V_{κ}2 | S | S | V | T | L | G |
| V_{κ}3 | S | T | V | T | L | G |

^{a}Numbering according to the structurally based scheme of Honegger & Plückthun (2001)

TABLE 5
Summary of biophysical characterization of scFv fragments

| | | | insoluble | oligo- | midpoint | |
|---|---|---|---|---|---|---|
| | | soluble | content | meric | [GdnHCl] | |
| | | soluble | content | meric | (M) | |
| scFv | CDR3 | yield^{c} | (%) | state^{d} | V_{H}^{e} | V_{L}^{e} |
| H1aκ3 | short / κ-like^{a} | 11.1 (1.7) | 10 | m,D,M | 1.8 | 2.8 |
| H1bκ3 | short / κ-like | 12.4 (1.9) | 20 | M | 2.4 | 3.0 |
| H2κ3 | short / κ-like | 2.6 (0.6) | 90 | M | 1.5 | 2.8 |
| H3κ3 | short / κ-like | 6.5 (=1) | 30 ± 10 | M | 2.8^{f} | |
| H4κ3 | short / κ-like | 2.6 (0.4) | 90 | M | 2.3 | 3.0 |
| H5κ3 | short / κ-like | 6.5 (1.0) | 50 | M | 2.2 | 3.0 |
| H6κ3 | short / κ-like | 5.2 (0.8) | 80 | M | 1.2 | 2.6 |
| H3κ1 | short / κ-like | 2.6 (0.4) | 50 | M | 2.8^{f} | |
| H3κ2 | short / κ-like | 2.6 (0.4) | 20 | M | 2.9 | 1.6 |
| H3κ3 | short / κ-like | 6.5 (=1) | 30 ± 10 | M | 2.8^{f} | |
| H3κ4 | short / κ-like | 5.2 (0.8) | 40 | M | 2.8 | 2.0 |

| | | | | | |
|---|---|---|---|---|---|
| H3λ1 | short / λ-like^{b} | 7.8 (1.2) | 40 | D, M | 3.0^{f} |
| H3λ2 | short / λ-like | 5.9 (0.9) | 10 | D, M | 2.9^{f} |
| H3λ3 | short / λ-like | 3.9 (0.6) | 10 | D, M | 2.8^{f} |

^{a}sequence of H-CDR3 (short, WGGDGFYAMDY) / L-CDR3 (κ-like: QQHYTTPPT)
^{b}sequence of H-CDR3 (short, WGGDGFYAMDY) / L-CDR3 (λ-like: QSYDSSLSGVV)
^{c}given in mg per 1 L bacteria at OD₅₅₀ of 10, and compared to in parenthesis to the soluble yield of H3κ3
^{d}oligomeric state in 50 mM sodium-phosphate (pH 7.0) and 500 mM NaCl with M monomer; D dimer; m multimer.
^{e}within the scFv fragment
^{f}only one transition is visible

TABLE 6
Framework usage in vivo and in vitro

| | | Framework usage of | | | |
|---|---|---|---|---|---|
| | | | 137 binders | | 250 |
| | | | from | Theoretical | binders |
| | Human | germline | Griffiths | distribution | from |
| | family | segments^{a} | library^{b} | of HuCAL^{c} | HuCAL^{d} |
| V_{H} | 1a and 1b | 24%^{g} | 13% | 12% | 16% |
| | 2 | 6% | 0% | 9% | 22% |
| | 3 | 43% | 74% | 10% | 36% |
| | 4 | 22% | 11% | 19% | 1% |
| | 5 | 4% | 1% | 18% | 13% |
| | 6 | 2%^{f} | 0% | 32% | 12% |
| V_{L} | κ1 | 25% | 7% | 16% | 13% |
| | κ2 | 12% | 47% | 16% | 5% |
| | κ3 | 9% | 2% | 16% | 17% |
| | κ4 | 1%^{f} | 0% | 16% | 12% |
| | λ1 | 9% | 28% | 12% | 13% |
| | λ2 | 8% | 4% | 12% | 11% |
| | λ3 | 14% | 9% | 12% | 28% |
| | other | 26% | 2% | — | — |

^{a}Taken from VBASE; 51 human germline segments for V_{H} and 76 for V_{L}.
^{b}Taken from Griffiths et al., (1994), originally 215 binders were sequenced but there are only 137 unique sequences. The Griffiths library is built from an in vitro rearranged germline bank, therefore the theoretical distribution is given by the percentage of germline segment, present in the human genome, as given in column 3.
^{c}Theoretical distribution is corrected for size of sublibaries and percentage of correct clones in the original HuCAL-1 scFv library (Knappik et al., (2000).
^{d}Taken from (Knappik et al., (2000).
^{e}including DP-21 (V_{H}7)
^{f}one germline segment

TABLE 7
Summary of yield and stability measurements

| | | Yield: | | Stability: | |
|---|---|---|---|---|---|
| | | normalized to wt^{a} | | ΔΔG_{N-U}(kJ / mol)^{b} | |
| name | abbreviation | 2C2 | 6B3 | 2C2 | 6B3 |
| wt | | =1 | =1 | =0 | =0 |
| Q5V | a | 1.7 | 2.6 | 2.4 | 2.9 |
| S16G | b | 1.8 | 2.3 | 6.2 | 7.3 |
| T58I | c | 1.0 | 0.9 | 7.9 | 6.8 |
| V72D | d | 3.2 | 1.8 | 0.1 | 2.2 |
| S76G | e | 2.1 | 1.5 | 3.7 | 3.5 |
| S90Y | f | 1.3 | 1.8 | −0.1 | 1.4 |
| | ab | 1.8 | 3.5 | 9.8 (8.6)^{c} | n.d.^{d} |
| | ce | 1.4 | 1.4 | 10.4 (11.6) | n.d. |
| | abce | 2.3 | 3.1 | 18.9 (19.6) | n.d. |
| | abcde | 3.3 | 3.7 | 19.5 (19.7) | n.d. |
| all | abcdef | 4.3 | 4.2 | 20.9 (19.6) | n.d. |
| P10A | g | 2.9 | 4.2 | 0.0 | 0.0 |
| P10A + V74F | gh | 1.9 | 1.7 | 0.5 | 0.4 |
| all + P10A | abcdefg | 3.5 | 2.1 | 16.8 (19.6) | n.d. |

^{a}yield of soluble protein after IMAC and ion-exchange column, normalized to yield of the respective wild-type scFv fragments 2C2 and 6B3. Absolute values: 2C2-wt: 1.2 ± 0.1 mg and 6B3-wt: 0.4 ± 0.1 mg per 1 L bacterial culture of an OD₅₅₀ of 10.
^{b}Absolute values of free energy of unfolding of wild-type scFv fragments: 2C2-wt: ΔG_{N-U} 51.3 kJ / mol and 6B3-wt: ΔG_{N-U = 42.4 kJ / mol}
^{c}in parentheses sum of the free energy contributions of the individual mutations to equilibrium stability
^{d}not determined because of low cooperativity (see text for details)

TABLE 8
Analysis of framework-1 subtype

| | | subtype-defining residues^{a} | | | subtype-correlated core residues^{a} | | | |
|---|---|---|---|---|---|---|---|---|
| name | subtype | H6^{b} | H7 | H10 | H19 | H74 | H78 | H93 |
| | I | Glu | Ser | Pro | Leu | Leu | Ala/Val/Ile/Leu | Leu/Met |
| | II | Glu | Ser | Gly | Leu | Val | Phe | Met |
| | III | Gln | Ser | any (Ala)^{c} | Leu/Val | Phe | Ala/Val | Leu |
| wt | III | Gln (100%)^{d} | Ser (84%) | Pro (8%) | Leu (56%) | Ile (1%) | Ile (8%) | Leu (63%) |
| P10A | III | Gln | Ser | Ala | Leu | Ile | Ile | Leu |
| P10A | III | Gln | Ser | Ala | Leu | Phe | Ile | Leu |
| I74F | | | | | | | | |

^{a}according to ref. (32)
^{b}using the numbering scheme of Honegger & Plilckthun (33)
^{c}Ala is used in 76% of subtype III sequences (32)
^{d}percentage use of specified amino acid in subtype III sequences, regardless of VH family (32)

### REFERENCES

Ausubel, F. M., Brent, R., Kingston, R. E., Moore, D. D., Sedman, J. G., Smith, J. A., & Struhl, K. eds. (1999). Current Protocols in Molecular Biology. New York: John Wiley and Sons.
Bird, R. E., Hardman, K. D., Jacobson, J. W., Johnson, S., Kaufman, B. M., Lee, S. M., Lee, T., Pope, S. H., Riordan, G. S. & Whitlow, M. (1988). Single-chain antigen-binding proteins. Science 242, 423-426.
Boothmann, H. and Plückthun, A. (1998). Selection for a periplasmic factor improving phage display and functional periplasmic expression. Nat. Biotechnol. 16, 376-380.
Brinkmann, U., Reiter, Y., Jung, S., Lee, B. & Pastan, I. (1993). A recombinant immunotoxin containing a disulfide-stabilized Fv fragment. Proc. Natl. Acad. Sci. U.S.A. 90, 7538-7542.
Buchner, J. & Rudolph, R. (1991). Renaturation, purification and characterization of recombinant Fab-fragments produced in Escherichia coli. Biotechnology (NY) 9, 157-162.
Carter, P., Presta, L., Gorman, C. M., Ridgway, J. B., Henner, D., Wong, W. L., Rowland, A. M., Kotts, C., Carver, M. E. & Shepard, H. M. (1992). Humanization of an anti-p185HER2 antibody for human cancer therapy. Proc. Natl. Acad. Sci. USA 89, 4285-4289.
Cook, G. P. & Tomlinson, I. M. (1995). The human immunoglobulin V-H repertoire. Immunology Today 16, 237-242.
de Wildt, R. M., Hoet, R. M. A., van Venrooij, W. J., Tomlinson, I. M. & Winter, G. (1999). Analysis of heavy and light chain pairings indicates that receptor editing shapes the human antibody repertoire. J. Mol. Biol. 285, 895-901.
Dooley, H., Grant, S. D., Harris, W. J. & Porter, A. J. (1998). Stabilization of antibody fragments in adverse environments. Biotechnol. Appl. Biochem. 28, 77-83.
Edwards, B. M., Main, S. H., Cantone, K. L., Smith, S. D., Warford, A. & Vaughan, T. J. (2000). Isolation and tissue profiles of a large panel of phage antibodies binding to the human adipocyte cell surface. J. Immunol. Meth. 245, 67-78.
Eigenbrot, C., Randal, M., Presta, L., Carter, P. & Kossiakoff, A. A. (1993). X-ray structures of the antigen-binding domains from three variants of humanized anti-p185HER2 antibody 4D5 and comparison with molecular modeling. J. Mol. Biol. 229, 969-995.
Fink, A. L. (1998). Protein aggregation: folding aggregates, inclusion bodies and amyloid. Fold. Des. 3, R9-23.
Forsberg, G., Forsgren, M., Jaki, M., Norin, M., Sterky, C., Enhorning, A., Larsson, K., Ericsson, M. & Bjork, P. (1997). Identification of framework residues in a secreted recombinant antibody fragment that control production level and localization in Escherichia coli. J. Biol. Chem. 272, 12430-12436.
Glockshuber, R., Malia, M., Pfitzinger, I. & Plückthun, A. (1992). A comparison of strategies to stabilize immunoglobulin Fv-fragments. Biochemistry 29, 1362-1367.
Griffiths, A. D., Williams, S. C., Hartley, O., Tomlinson, I. M., Waterhouse, P., Crosby, W. L., Kontermann, R. E., Jones, P. T., Low, N. M., Allison, T. J., Prospero, T. D., Hoogenboom, H. R., Nissim, A., Cox, J. P. L., Harrison, J. L., Zaccolo, M., Gherardi, E. & Winter, G. (1994). Isolation of high affinity human antibodies directly from large synthetic repertoires. EMBO J. 13, 3245-3260.
Hanes, J., Jermutus, L., Weber-Bornhauser, S., Bosshard, H. R., and Plückthun, A. (1998) Ribosome display efficiently selects and evolves high-affinity antibodies in vitro from immune libraries, Proc. Natl. Acad. Sci. USA 95, 14130-14135.
Hanes, J., Schaffitzel, C., Knappik, A. & Plückthun, A. (2000). Picomolar affinity antibodies from a fully synthetic naive library selected and evolved by ribosome display: Nat. Biotechnol. 18, 1287-1292.
Harris, J. R., Plückthun, A. & Zahn, R. (1994). Transmission electron microscopy of GroEL, GroES, and the symmetrical GroEL/ES complex. J. Struct. Biol. 112, 216-230.
Henikoff, S. & Henikoff, J. G. (1992). Amino acid substitution matrices from protein blocks. Proc. Natl. Acad. Sci. USA 89, 10915-10919.
Holt, L. J., Bussow, K., Walter, G. & Tomlinson, I. M. (2000). By-passing selection: direct screening for antibody-antigen interactions using protein arrays. Nucleic Acids Res. 28, E72.
Honegger, A. & Plückthun, A. (2001 a). The influence of the buried glutamine or glutamat residue in position 6 on the structure of immunoglobuline variable domains. J. Mol. Biol., in press.
Honegger, A. & Plückthun, A. (2001b). Yet another numbering scheme for immunoglobulin variable domains: An automatic modeling and analysis tool. J. Mol. Biol., in press.
Huston, J. S., Levinson, D., Mudgett-Hunter, M., Tai, M. S., Novotny, J., Margolies, M. N., Ridge, R. J., Bruccoleri, R. E., Haber, E., Crea, R. & Oppermann, H. (1988). Protein engineering of antibody binding sites: recovery of specific activity in an anti-digoxin single-chain Fv analogue produced in Escherichia coli. Proc. Natl. Acad. Sci. USA 85, 5879-5883.
Jäger, M., Gehrig, P. & Plückthun, A. (2001). The scFv fragment of the antibody hu4D5-8: Evidence for early premature domain interaction in refolding. J. Mol. Biol. 305, 1111-1129.
Jäger, M. & Plückthun, A. (1999a). Domain interactions in antibody Fv and scFv fragments: effects on unfolding kinetics and equilibria. FEBS Lett. 462, 307-312.
Jäger, M. & Plückthun, A. (1999b). Folding and assembly of an antibody Fv fragment, a heterodimer stabilized by antigen. J. Mol. Biol. 285, 2005-2019.
Jung, S., Honegger, A. & Plückthun, A. (1999). Selection for improved protein stability by phage display. J. Mol. Biol. 294, 163-180.
Jung, S., Spinelli, S., Schimmele, B., Honegger, A., Pugliese, L., Cambillau, C. & Plückthun, A. (2001). The importance of framework residues H6, H7 and H10 in antibody heavy chains: experimental evidence for a new structural subclassification of antibody VH domain. J. Mol. Biol., in press.
Kabat, E. A., Wu, T. T., Perry, H. M., Gottesmann, K. S. & Foeller, C. (1991). Variable region heavy chain sequences. In Sequences of Proteins of Immunological Interest. NIH Publication No. 91-3242, National Technical Information Service (NTIS).
Kipriyanov, S. M., Moldenhauer, G., Martin, A. C., Kupriyanova, O. A. & Little, M. (1997). Two amino acid mutations in an anti-human CD3 single chain Fv antibody fragment that affect the yield on bacterial secretion but not the affinity. Protein Eng. 10, 445-453.
Knappik, A., Ge, L., Honegger, A., Pack, P., Fischer, M., Wellnhofer, G., Hoess, A., Wolle, J., Plückthun, A. & Virnekas, B. (2000). Fully synthetic human combinatorial antibody libraries (HuCAL) based on modular consensus frameworks and CDRs randomized with trinucleotides. J. Mol. Biol. 296, 57-86.
Knappik, A. & Plückthun, A. (1995). Engineered turns of a recombinant antibody improve its in vivo folding. Protein Eng. 8, 81-89.
Koradi, R., Billeter, M. & Wüthrich, K. (1996). MOLMOL: a program for display and analysis of macromolecular structures. J. Mol. Graph. 14, 51-55, 29-32.
Krebber, A., Bornhauser, D., Burmester, J., Honegger, A., Willuda, J., Bossard, H. R. & Plückthun, A. (1997). Reliable cloning of functional antibody varable domains from hybridomas and spleen cell repertoires employing a reengineered phage display system. J. Immunol. Meth. 201, 35-55.
Lindner, P., Bauer, K., Krebber, A., Nieba, L., Kremmer, E., Krebber, C., Honegger, A., Klinger, B., Mocikat, R. and Plückthun, A. (1997). Specific detection of his-tagged proteins with recombinant anti-His tag scFv-phosphatase or scFv-phage fusions. Biotechniques 22, 140-149.
Liu, N., Deillon, C., Klauser, S., Gutte, B. and Thomas, R. M. (1998). Synthesis, physiochemical characterization, and crystallization of a putative retro-coiled coil. Protein Sci. 7, 1214-1220.
Myers, J. K., Pace, C. N. & Scholtz, J. M. (1995). Denaturant m values and heat capacity changes: relation to changes in accessible surface areas of protein unfolding. Prot. Sci. 4, 2138-2148.
Nakamura, H. (1996). Roles of electrostatic interaction in proteins. Q. Rev. Biophys. 29, 1-90.
Nieba, L., Honegger, A., Krebber, C. & Plückthun, A. (1997). Disrupting the hydrophobic patches at the antibody variable/constant domain interface: improved in vivo folding and physical characterization of an engineered scFv fragment. Protein Eng. 10, 435-444.
Ohage, E. C., Wirtz, P., Barnikow, J. & Steipe, B. (1999). Intrabody construction and expression. II. A synthetic catalytic Fv fragment. J. Mol. Biol. 291, 1129-1134.
Pace, C. N. (1990). Measuring and increasing protein stability. Trends Biotechnol. 8, 93-98.
Pace, C. N. & Scholtz, J. M. (1997) in Protein Structure, A Practical Approach (Creighton, ed), pp. 299-321, Oxford University Press, New York.
Pini, A., Viti, F., Santucci, A., Carnemolla, B., Zardi, L., Neri, P. & Neri, D. (1998). Design and use of a phage display library. Human antibodies with subnanomolar affinity against a marker of angiogenesis eluted from a two-dimensional gel. J. Biol. Chem. 273, 21769-21776.
Plückthun, A., Krebber, A., Horn, U., Knüpfer, U., Wenderoth, R., Nieba, L., Proba, K. & Riesenberg, D. (1996). Producing antibodies in Escherichia coli: From PCR to fermentation. In Antibody Engineering, A Practical Approach (Mc Cafferty, J., Hoogenboom, H. R. & Chiswell, D. J., eds.), pp. 203-252. Oxford University Press, New York.
Raffen, R., Dieckman, L. J., Szpunar, M., Wunschl, C., Pokkuluri, P. R., Dave, P., Wilkens Stevens, P., Cai, X., Schiffer, M. & Stevens, F. J. (1999). Physicochemical consequences of amino acid variations that contribute to fibril formation by immunoglobulin light chains. Protein Sci. 8, 509-517.
Rodrigues, M. L., Shalaby, M. R., Werther, W., Presta, L. & Carter, P. (1992). Engineering a humanized bispecific F(ab′)2 fragment for improved binding to T cells. Int. J. Cancer Suppl. 7, 45-50.
Sambrook, J., Fritsch, E. F. and Maniatis, T. (1989) Molecular Cloning: A laboratory manual, Cold Spring Harbor Laboratory Press, Cold Spring Harbor, USA.
Saul, F. A. & Poljak, R. J. (1993). Structural patterns at residue positions 9, 18, 67 and 82 in the VH framework regions of human and murine immunoglobulins. J. Mol. Biol. 230, 15-20.
Skerra, A. & Plückthun (1988). Assembly of a functional immunoglobulin Fv fragment in Escherichia coli. Science 240, 1038-1041.
Söderlind, E., Strandberg, L., Jirholt, P., Kobayashi, N., Alexeiva, V., Aberg, A. M., Nilsson, A., Jansson, B., Ohlin, M., Wingren, C., Danielsson, L., Carlsson, R. & Borrebaeck, C. A. (2000). Recombining germline-derived CDR sequences for creating diverse single-framework antibody libraries. Nat. Biotechnol. 18, 852-856.
Steipe, B., Schiller, B., Plückthun, A. & Steinbacher, S. (1994). Sequence statistics reliably predict stabilizing mutations in a protein domain. J. Mol. Biol. 240, 188-192.
Tomlinson, I. M., Walter, G., Marks, J. D., Llewelyn, M. B. & Winter, G. (1992). The repertoire of human germline VH sequences reveals about fifty groups of VH segments with different hypervariable loops. J. Mol. Biol. 227, 776-798.
Vaughan, T. J., Williams, A. J., Pritchard, K., Osbourn, J. K., Pope, A. R., Earnshaw, J. C., McCafferty, J., Hodits, R. A., Wilton, J. & Johnson, K. S. (1996). Human antibodies with sub-nanomolar affinities isolated from a large non-immunized phage display library. Nat. Biotechnol. 14, 309-314.
Willuda, J., Honegger, A., Waibel, R., Schubiger, P. A., Stahel, R., Zangemeister-Wittke, U. & Plückthun, A. (1999). High thermal stability is essential for tumor targeting of antibody fragments: engineering of a humanized anti-epithelial glycoprotein-2 (epithelial cell adhesion molecule) single-chain Fv fragment. Cancer Res. 59, 5758-5767.
Wirtz, P. & Steipe, B. (1999). Intrabody construction and expression III: engineering hyperstable V(H) domains. Protein Sci. 8, 2245-2250.
Wörn, A. & Plückthun, A. (1999). Different equilibrium stability behavior of ScFv fragments: identification, classification, and improvement by protein engineering. Biochemistry 38, 8739-8750.
Wörn, A. & Plückthun, A. (2001). Stability engineering of antibody single-chain Fv fragments. J. Mol. Biol. 305, 989-1010.
Zouali, M. & Theze, J. (1991). Probing VH gene-family utilization in human peripheral B cells by in situ hybridization. J. Immunol. 146, 2855-2864.

1. An isolated polypeptide comprising a V_{H} domain selected from the group consisting of (i) a V_{H} domain belonging to the V_{H}1a subclass, wherein said V_{H} domain comprises an amino acid residue F at position 29 and/or L at position 89; (ii) a V_{H} domain belonging to the V_{H}1b subclass, wherein said V_{H} domain comprises the amino acid residue L at position 89; (iii) a V_{H} domain belonging to the V_{H}2 subclass, wherein said V_{H} domain comprises at least one amino acid residue selected from the group consisting of G at position 16, V at position 44, A at position 47, G at position 76, F at position 78, Y at position 90, R at position 97, E at position 99, wherein if R is at position 97, then E is at position 99; (iv) a V_{H} domain belonging to the V_{H}4 subclass, wherein said V_{H} domain comprises at least one amino acid residue selected from the group consisting of G at position 16, A at position 47, F at position 78, Y at position 90, R at position 97, and E at position 99, wherein if R is at position 97, then E is at position 99; (v) a V_{H} domain belonging to the V_{H}5 subclass, wherein said V_{H} domain comprises at least one amino acid residue selected from the group consisting of L at position 89, R at position 97, and E at position 99, wherein if R is at position 97, then E is at position 99; and (vi) a V_{H} domain belonging to the V_{H}6 subclass, wherein said V_{H} domain comprises at least one amino acid residue selected from the group consisting of V at position 5, G at position 16, I at position 58, F at position 78, Y at position 90 and R at position 97, and E at position 99, wherein if R is at position 97, then E is at position 99.

2. An isolated polypeptide according to claim 1, comprising a V_{H} domain belonging to the V_{H}1a subclass, wherein said V_{H} domain comprises an amino acid residue F at position 29 and/or L at position 89.

3. An isolated polypeptide according to claim 1, comprising a V_{H} domain belonging to the V_{H}1b subclass, wherein said V_{H} domain comprises the amino acid residue L at position 89.

4. An isolated polypeptide according to claim 1, comprising a V_{H} domain belonging to the V_{H}2 subclass, wherein said V_{H} domain comprises at least one amino acid residue selected from the group consisting of G at position 16, V at position 44, A at position 47, G at position 76, F at position 78, Y at position 90, R at position 97, E at position 99, wherein if R is at position 97, then E is at position 99.

5. An isolated polypeptide according to claim 1, comprising a V_{H} domain belonging to the V_{H}4 subclass, wherein said V_{H} domain comprises at least one amino acid residue selected from the group consisting of G at position 16, A at position 47, F at position 78, Y at position 90, R at position 97, and E at position 99, wherein if R is at position 97, then E is at position 99.

6. An isolated polypeptide according to claim 1, comprising a V_{H} domain belonging to the V_{H}5 subclass, wherein said V_{H} domain comprises at least one amino acid residue selected from the group consisting of L at position 89, R at position 97, and E at position 99, wherein if R is at position 97, then E is at position 99.

7. An isolated polypeptide according to claim 1, comprising a V_{H} domain belonging to the V_{H}6 subclass, wherein said V_{H} domain comprises at least one amino acid residue selected from the group consisting of V at position 5, G at position 16, I at position 58, F at position 78, Y at position 90 and R at position 97, and E at position 99, wherein if R is at position 97, then E is at position 99.

8. An antibody or functional fragment thereof comprising a V_{H} domain according to claim 1.

9. A library of antibodies or functional fragments thereof comprising one or more antibodies or functional fragments thereof according to claim 8.

10. An isolated nucleic acid sequence encoding a polypeptide selected from the group consisting of (i) a polypeptide comprising a V_{H} domain belonging to the V_{H}1a subclass, wherein said V_{H} domain comprises an amino acid residue F at position 29 and/or L at position 89; (ii) a polypeptide comprising a V_{H} domain belonging to the V_{H}1b subclass, wherein said V_{H} domain comprises the amino acid residue L at position 89; (iii) a polypeptide comprising a V_{H} domain belonging to the V_{H}2 subclass, wherein said V_{H} domain comprises at least one amino acid residue selected from the group consisting of G at position 16, V at position 44, A at position 47, G at position 76, F at position 78, Y at position 90, R at position 97, E at position 99, wherein if R is at position 97, then E is at position 99; (iv) a polypeptide comprising a V_{H} domain belonging to the V_{H}4 subclass, wherein said V_{H} domain comprises at least one amino acid residue selected from the group consisting of G at position 16, A at position 47, F at position 78, Y at position 90, R at position 97, and E at position 99, wherein if R is at position 97, then E is at position 99; (v) a polypeptide comprising a V_{H} domain belonging to the V_{H}5 subclass, wherein said V_{H} domain comprises at least one amino acid residue selected from the group consisting of L at position 89, R at position 97, and E at position 99, wherein if R is at position 97, then E is at position 99; and (vi) a polypeptide comprising a V_{H} domain belonging to the V_{H}6 subclass, wherein said V_{H} domain comprises at least one amino acid residue selected from the group consisting of V at position 5, G at position 16, I at position 58, F at position 78, Y at position 90 and R at position 97, and E at position 99, wherein if R is at position 97, then E is at position 99.

11. A vector comprising a nucleic acid sequence corresponding to the nucleic acid sequence according to claim 10.

12. A host cell harboring a nucleic acid sequence corresponding to the nucleic acid sequence according to claim 10.

13. A method for producing a V_{H} domain or an antibody or a functional fragment thereof comprising the step of expressing an isolated nucleic acid sequence according to claim 10.

14. A method for obtaining an isolated nucleic acid sequence, comprising the step of (i) substituting, in a nucleic acid sequence that encodes a V_{H}1a subclass domain, at least one codon that encodes an amino acid residue selected from the group consisting of F at position 29 and L at position 89; or (ii) substituting, in a nucleic acid sequence that encodes a V_{H}1b subclass domain, a codon that encodes the amino acid residue L at position 89; or (iii) substituting, in a nucleic acid sequence that encodes a V_{H}2 subclass domain, at least one codon that encodes an amino acid residue selected from the group consisting of G at position 16, V at position 44, A at position 47, G at position 76, F at position 78, R at position 97, and E at position 99, wherein if R is at position 97, then E is at position 99; or (iv) substituting, in a nucleic acid sequence that encodes a V_{H}2 subclass domain, a codon that encodes the amino acid residue Y at position 90; or (v) substituting, in a nucleic acid sequence that encodes a V_{H}4 subclass domain, at least one codon that encodes an amino acid residue selected from the group consisting of G at position 16, V at position 44, A at position 47, G at position 76, F at position 78, R at position 97, and E at position 99, wherein if R is at position 97, then E is at position 99; or (vi) substituting, in a nucleic acid sequence that encodes a V_{H}4 subclass domain, a codon that encodes the amino acid residue Y at position 90; or (vii) substituting, in a nucleic acid sequence that encodes a V_{H}5 subclass domain, at least one codon that encodes an amino acid residue selected from the group consisting of R at position 77, L at position 89, R at position 97, and E at position 99, wherein if R is at position 97, then E is at position 99; or (viii) substituting, in a nucleic acid sequence that encodes a V_{H}6 subclass domain, at least one codon that encodes an amino acid residue selected from the group consisting of V at position 5, G at position 16, V at position 44, I at position 58, D at position 72, G at position 76, F at position 78, R at position 97, and E is at position 99, wherein if R is at position 97, then E is at position 99; or (ix) substituting, in a V_{H}6 subclass domain, a codon that encodes the amino acid residue Y at position 90.

15. A method according to claim 14, wherein 2 or more codons are substituted in said nucleic acid sequence.

16. A method according to claim 14, further comprising the steps of:
(i) identifying for said domain the corresponding amino acid consensus sequence selected from the group of V_{H} consensus sequences consisting of V_{H}1a, V_{H}1b, V_{H}2, V_{H}4, V_{H}5, and V_{H}6;
(ii) substituting one or more codons corresponding to amino acid residues of said consensus sequence into a corresponding position(s) in said nucleic acid sequence of said domain.

17. A method of obtaining a polypeptide, comprising the step of expressing a nucleic acid sequence according to claim 14.

18. A method for constructing a library of antibodies or functional fragments thereof comprising the steps of: (i) obtaining at least one nucleic acid sequence according to claim 14; and (ii) diversifying said obtained nucleic acid sequence to generate a population of diversified nucleic acid sequences, wherein said diversified nucleic acid sequences can be expressed for generating and screening of antibody libraries comprising diversified V_{H} domains.

19. An isolated polypeptide comprising a V_{L} domain selected from the group consisting of (i) a V_{L} domain belonging to the V_{L}κ2 subclass, wherein said V_{L} domain comprises the amino acid residue R at position 18, and wherein if R is at position 18, then T is at position 92; and (ii) a V_{L} domain belonging to the V_{L}λ1 subclass, wherein said V_{L} domain comprises the amino acid residue K at position 47.

20. An isolated polypeptide according to claim 19, comprising a V_{L} domain belonging to the V_{L}κ2 subclass, wherein said V_{L} domain comprises the amino acid residue R at position 18, and wherein if R is at position 18, then T is at position 92.

21. An isolated polypeptide according to claim 19, comprising a V_{L} domain belonging to the V_{L}λ1 subclass, wherein said V_{L} domain comprises the amino acid residue K at position 47.

22. An antibody or a functional fragment thereof comprising a V_{L} domain according to claim 19.

23. A library of antibodies or functional fragments thereof comprising one or more antibodies or functional fragments thereof according to claim 22.

24. An isolated nucleic acid molecule encoding a polypeptide selected from the group consisting of (i) a polypeptide comprising a V_{L} domain belonging to the V_{L}κ2 subclass, wherein said V_{L} domain comprises the amino acid residue R at position 18, and wherein R is at position 18, then T is at position 92; and (ii) a polypeptide comprising a V_{L} domain belonging to the V_{L}λ1 subclass, wherein said V_{L} domain comprises the amino acid residue K at position 47.

25. A vector comprising a nucleic acid sequence corresponding to the nucleic acid sequence according to claim 24.

26. A host cell harbouring a nucleic acid sequence molecule corresponding to the nucleic acid sequence according to claim 24.

27. A method for producing a V_{L} domain or an antibody or a functional fragment thereof comprising the step of expressing an isolated nucleic acid sequence according to claim 24.

28. A method for obtaining a nucleic acid sequence, comprising the step of (i) substituting, in a nucleic acid sequence that encodes a V_{L}κ2 subclass domain, at least one codon that encodes an amino acid residue selected from the group consisting of S at position 12, Q at position 45, and R at position 18, and wherein if R is at position 18, then T is at position 92; or (ii) substituting, in a nucleic acid sequence that encodes a V_{L}λ1 subclass domain, at least one codon that encodes the amino acid residue K at position 47; or (iii) substituting, in a nucleic acid sequence that encodes a V_{L}λ1 domain, at least three codons that encode the amino acid residues S at position 7, P at position 8, and S at position 9, respectively; or (iv) substituting, in a nucleic acid sequence that encodes a V_{L}λ2 domain, at least three codons that encode the amino acid residues S at position 7, P at position 8, and S at position 9, respectively; or (v) substituting, in a nucleic acid sequence that encodes a V_{L}λ3 domain, at least three codons that encode the amino acid residues S at position 7, P at position 8, and S at position 9, respectively.

29. A method according to claim 28, wherein 2 or more codons are substituted in said nucleic acid sequence.

30. A method according to claim 28, further comprising the steps of:
(i) identifying for said domain the corresponding amino acid consensus sequence selected from the group of V_{L} consensus sequences consisting of V_{L}λ2, V_{L}λ1 V_{L}λ2, and V_{L}λ3; and
(ii) substituting one or more codons corresponding to amino acid residues of said consensus sequence into a corresponding position(s) in said nucleic acid sequence of said domain.

31. A method of obtaining a polypeptide, comprising the step of expressing a nucleic acid sequence according to claim 24.

32. A method for constructing a library of antibodies or functional fragments thereof, comprising the steps of: (i) obtaining at least one nucleic acid sequence according to claim 24; and (ii) diversifying said obtained nucleic acid sequence to generate a population of diversified nucleic acid sequences, wherein said diversified nucleic acid sequences can be expressed for generating and screening of antibody libraries comprising said diversified VH domains.

33. An antibody or a functional fragment thereof comprising a polypeptide of claim 1 and a polypeptide comprising a V_{L} domain selected from the group consisting of (i) a V_{L} domain belonging to the V_{L}κ2 subclass, wherein said V_{L} domain comprises the amino acid R at position 18, and wherein if R is at position 18, then T is at position 92; and (ii) a V_{L} domain belonging to the V_{L}λ1 subclass, wherein said V_{L} domain comprises the amino acid residue K at position 47.


SEQUENCE LISTING (19 sequences)

SEQ ID NO: 1 - protein, 17 aa, artificial sequence
  synthetic construct
YNHEADMLIRNWLYSDV

SEQ ID NO: 2 - protein, 4 aa, artificial sequence
  synthetic construct
DYKD

SEQ ID NO: 3 - protein, 11 aa, artificial sequence
  synthetic construct
QSYDSSLSGVV

SEQ ID NO: 4 - protein, 7 aa, artificial sequence
  synthetic construct
DYKDDDD

SEQ ID NO: 5 - protein, 11 aa, artificial sequence
  synthetic construct
WGGDGFYAMDY

SEQ ID NO: 6 - protein, 8 aa, artificial sequence
  synthetic construct
YDSSLSGV

SEQ ID NO: 7 - protein, 19 aa, artificial sequence
  synthetic construct
QRGHYGKGYKGFNSGFFDF

SEQ ID NO: 8 - protein, 7 aa, artificial sequence
  synthetic construct
QYYNIPT

SEQ ID NO: 9 - protein, 26 aa, artificial sequence
  synthetic construct
CDAFRSEKSRQELNTIASKPPRDHVF

SEQ ID NO: 10 - protein, 11 aa, artificial sequence
  synthetic construct
SYFISFFSFDY

SEQ ID NO: 11 - protein, 9 aa, artificial sequence
  synthetic construct
SYDSGFSTV

SEQ ID NO: 12 - protein, 9 aa, artificial sequence
  synthetic construct
QQHYTTPPT

SEQ ID NO: 13 - protein, 108 aa, artificial sequence
  synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAWGQGTLVTVSS

SEQ ID NO: 14 - protein, 108 aa, artificial sequence
  synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCAWGQGTLVTVSS

SEQ ID NO: 15 - protein, 108 aa, artificial sequence
  synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAWGQGTLVTVSS

SEQ ID NO: 16 - protein, 108 aa, artificial sequence
  synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAWGQGTLVTVSS

SEQ ID NO: 17 - protein, 109 aa, artificial sequence
  synthetic construct
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCAWGQGTLVTVSS

SEQ ID NO: 18 - protein, 107 aa, artificial sequence
  synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAWGQGTLVTVSS

SEQ ID NO: 19 - protein, 111 aa, artificial sequence
  synthetic construct
QVQLQQSGPGLVKPSQTLSLTCAISGDSVSSNSAAWNWIRQSPGRGLEWLGRTYYRSKWYNDYAVSVKSRITINPDTSKNQFSLQLNSVTPEDTAVYYCAWGQGTLVTVSS